(19) United States
(12) Patent Application Publication
(10) Pub. No.: US 2011/0190805 A1
(43) Pub. Date: Aug. 4, 2011

(54) Intraurethral Retainer and Keeper For Facilitating Penile Fixation and/or Occlusion

(75) Inventor: Reinhard Carl Huber, Riverside, CA (US)

(73) Assignee: Reinhard Carl Huber, Riverside, CA (US)

(21) Appl. No.: 12/697,472

(22) Filed: Feb. 1, 2010

(51) Int. Cl. A61M 29/00

(52) U.S. Cl. 606/191

(57) ABSTRACT

An intraurethral retainer and a keeper that cooperatively function to achieve retention within the confines of the fossa navicularis of the human penis for the secure and reliable fixation of various engaging devices to the penis and/or for occlusion of the urethra, or to alone without an engaging device accomplish fixation and/or occlusion of the penis, without distending or exerting uncomfortable pressure or friction on the constriction of the urethral orifice during their insertion or removal and without of their own accord, or by their mere presence, exerting uncomfortable pressure or friction on the urethral wall while in place, in the absence of any other force that may be exerted thereon.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of Invention

There are many applications, both existing or novel as disclosed herein, which occasion the need for fixating the human penis to an object or device, whether it be to spatially orient the penis relative to the body in some particular position or state of animation in order to facilitate a procedure or to produce a therapeutic effect, or to secure the penis itself relative to the placement of some object or device either outwardly thereon or internally within the urethra, here the word “fixation” and its derivatives meaning any action or reaction that restricts the movement of one object with respect to another without regard to the degree of freedom of movement between the objects that that action or reaction may still permit. Likewise there are numerous applications, both existing and potentially novel as disclosed herein, which occasion the need for occluding the male urethra, whether it be to prevent or regulate the escape of bodily fluids like urine or semen, or to aid in the intraurethral administration of medicines or other agents like irrigating fluids or x-ray contrast mediums, here the word “occlusion” and its derivatives meaning any action or reaction that artificially restricts or otherwise influences the natural flow of fluids through an otherwise open lumen, fluids being any liquid or gas.

Often these two basic needs go hand-in-hand and occur simultaneously, whether or not actually thought of in separate terms, being that technically occlusion of any lumen is generally not possible without involving fixation of that lumen in some manner to whatever is occluding it. Some of the most common known applications exhibiting these two basic and often intertwined needs include corrective, exploratory or cosmetic surgery, physical therapy, management of urinary incontinence, prosthetic attempts to treat erectile dysfunction and male contraception, all in the general health care field. Lesser known, or novel as disclosed herein, the applications can also include erotic stimulation or masturbation aids and vanity products like body jewelry for sexual expression and adult amusement in the entertainment and personal consumer goods fields. The prior art in these fields has often had serious shortcomings and limitations, or has been non-existent. It is an objective of this invention to overcome these shortcomings and limitations and fulfill these needs in a manner never before contemplated.

(2) Description of Related Art

The prior art, as it relates to methods of fixing the penis to objects or devices and/or occluding the penis in attempting to fulfill the above needs, can be grouped into three broadly distinct categories:

In the first general category of penile fixation and/or occlusion are methods and devices that rely on direct physical attachment to the penis. These include suturing, tying, taping, gluing with the use of adhesive compounds, permanent surgical implantation, or permanently piercing the urethra or body of the penis to effect reversible attachment. For example, U.S. Pat. No. 6,743,209 B2 granted to Brown et al. discloses a urethral catheter with integral anchoring means designed specifically to facilitate suturing it to the penile prepuce or other epidermal areas of the penis surrounding the catheter. Alternatively, the invention also facilitates tying or taping the catheter to the penis. For similar purposes, U.S. Pat. No. 5,630,429 granted to Dann discloses a male incontinence device incorporating a sheath or cup that fits over the glans of the penis and is held there by adhesive. In other more diverse applications typical of the prior art in this category, U.S. Pat. No. 6,186,943 B1 granted to Pan discloses a penis support device (a prosthesis of sorts to aid sexual intercourse) that employs a “rope” that is tied around the glans of the penis to hold it to an external support member in order to keep the penis stiff and elongated. Utilizing basically the same method of attachment for a different purpose, U.S. Pat. No. 6,416,460 B1 granted to Jochum discloses a penis extension device that is intended to enlarge the male member by placing it under prolonged long-term traction via a “small band” that similarly fits around the base of the glans below the corona. In yet another application, U.S. Pat. No. 6,736,142 B2 granted to Sanchez Gomez discloses a protective tube and harness that is intended for use as a male chastity device. The tube assembly consists of two elements: a “fixation piece” that is securely fastened to the penis by wrapping both with adhesive tape, and a covering “safety tube” that is then slipped over and padlocked to the fixation piece to prevent removal of the tape. In all of these references and those that follow, the terminology of the original disclosure is used in their descriptions.

Still in the first category of direct physical attachment, examples of surgical implantation are numerous and include prosthetic devices to achieve a semblance of erection and artificial sphincters or valves to control urinary incontinence. These are usually extreme methods of last resort sharing the same purposes as the less invasive alternatives described herein and which might not be necessary if the latter were more effective in staying attached or remaining in place. Their need for surgical implantation is itself descriptive enough and they are therefore only mentioned for completeness with no specific references given.

As a final example, the prior art as it relates to this first category of direct physical attachment also includes the field of body piercing jewelry and personal adornment. Genital body piercing, for the purpose of attaching jewelry and for sexual enhancement, has occurred throughout history and has recently undergone a revival such that it is now within the cultures (some might even say mainstream) of many of today's modern societies and requires no specific reference. Popular among many men is the so-called “Prince Albert” which is a permanent body piercing from within the urethra to the underside of the penis below the glans, or the “Ampallang” or “Apadravya” which are the common names given to permanent piercings directly through the body of the glans penis, in all cases accompanied by the insertion of a ring, barbell or other appliance through the hole that was created, presumably for adornment or for aiding in sexual activities. Abundant descriptions of these genital piercings and the various jewelry or appliances used therewith can easily be found by searching the Internet.

All of the above methods in the first category of direct physical attachment have serious disadvantages and shortcomings that sharply limit their applicability, effectiveness and appeal and are mentioned specifically because in many instances they could be improved or replaced by the current invention. Sutures, while relatively secure, are painful and psychologically abhorrent to many patients and cause visible scarring, besides carrying a serious risk of infection and the danger of being torn out unless deeply embedded and liberally applied in number. Surgical implants are very costly and likewise inherently abhorrent and risky, and usually entail even more pain and scarring. Adhesive tapes and compounds, which by their nature must be removable, have limited strength and are good for only relatively short periods before they loose their grip, besides carrying the risk of allergic reaction. Methods that rely on inelastic ligatures like cords, straps and tapes, or dimensionally fixed coverings like cups and tubes, to wrap and tie or glue the penis to the device being contemplated are of equally limited usefulness and effectiveness, in large part due to their inability to adequately cope with the ever changing size and rigidity of the penis between its flaccid and erect states. Lastly, there are many men who wish they could have something ornamentally or functionally akin to a Prince Albert, Ampallang or Apadravya body piercing for its aesthetic and sexually enhancing benefits, but are dissuaded by the pain and permanent disfigurement it would cause and by the time needed for healing and for the period of stretching and adjustment to the inserted appliance. Adaptation of the invention disclosed herein could avoid all these disadvantages and shortcomings and provide a better alternative.

A second broad category in the prior art involves methods and devices that rely primarily on the principles of pressure and friction to maintain a grip on either the urethra or the outer body of the penis. This includes both externally applied and intraurethral means; intraurethral generally meaning either partially or wholly within the urethra and in this category the discussion of the intraurethral means being limited to those means that are positioned and act internally within the shaft or corpus cavernosum of the penis by deep urethral insertion and/or which make no attempt, either by default or by specific intent, to take advantage of the fossa navicularis, a natural enlargement of the male urethra that occurs more proximally inside the glans of the penis.

Applications in the second category that rely on externally applied pressure and friction are numerous. For example, U.S. Pat. Application Pub. No. US 2003/0018321 A1 discloses a male catheter-securing device that functions similarly to “Chinese handcuffs” or “finger-traps” to grab the penis externally and accomplish its purpose. In a more unusual application, U.S. Pat. No. 5,599,275 granted to France discloses a penis stretching apparatus consisting simply of a pair of hinged semicircular metal weights that encircle the shaft of the penis and are held together by either a metal ring, or alternatively by a rubber band that is intended to allow conformity between the flaccid and erect states. Similarly, but somewhat more cognitive of comfort and blood circulation concerns, U.S. Pat. No. 6,033,374 granted to Miller, Jr. discloses a penile traction device intended for physical therapy to mitigate scarring and contracture at incisions following surgery, as well as for penile lengthening and other pursuits. The Miller device also employs weights, but with a more gentle foam pressure-dissipating friction pad that is wrapped around the penile shaft and then attached to the weight. Probably no other application has however received as much attention as the many attempts to control urinary incontinence through the direct use of constricting external clamps and bands. U.S. Pat. No. 6,609,522 B2 granted to Cheng et al. is just one recent example.

A principal drawback of all such external pressure and friction devices and methods, despite all the counteractive and mitigating measures that have been tried, is their tendency to exert uncomfortable and possibly injurious pressure and to become too loose when the penis becomes flaccid, or conversely too tight when the penis becomes erect, necessitating frequent repositioning and adjustment. In many cases, adaptation of the present invention to these applications, in lieu of the devices and methods used until now, might be preferable.

Examples of intraurethrally-applied pressure or friction dependent methods in the second category are more diverse. U.S. Pat. No. 2,696,209 granted to Varaney discloses an internal prophylactic protector (for contraception) consisting of a tubular element with an attached folded up sack that is inserted past the glans and then squeezed deep into the urethra by external manipulation. Necessarily being small enough to get past the external urethral orifice (a pronounced constriction that is substantially narrower than any other part of the urethra) and to be positioned in this manner, it is by default held in place by only light friction and its ability to stay in place and function as intended is correspondingly limited. Somewhat more aggressively, U.S. Pat. No. 4,968,294 granted to Salama discloses a urinary control valve that follows the same principles of retention but incorporates external serrations to deliberately increase friction besides reducing leakage. Still, it too must be small enough to squeeze past the external urethral orifice, which again limits its ability to seal and stay in place once at its intended location. Even more aggressively, U.S. Pat. No. 4,457,299 granted to Cornwell discloses an incontinence control device consisting of a compressible “capsule” of elliptical cross section that is inserted deep into the urethra. The spring-loaded device operates on principles that require the urethra to be kept in a continuous state of lateral distension via a pre-stressed integral spring and additionally incorporates a matt, serrated or striated (his word) surface to provide friction and reduce leakage. Nevertheless, the description also includes optional auxiliary attachment to a second urethral insert that is positioned in the even deeper prostatic region, indicating that alone the device might still not be secure enough to resist expulsion. Using a more popular approach to the problems of slippage and leakage, U.S. Pat. No. 4,934,999 granted to Bader discloses a closure device consisting of a single tubular element, or alternatively multiple articulated elements, enveloped by an inflatable membrane that is charged by a hand pump to exert lateral pressure and seal the device against the urethral wall in a fixed relationship. The device is intended to control incontinence, act as a contraceptive and also be used for “enabling artificial erection”. Also relying on fluid pressure to inflate a device and thereby achieve a fixed seal within the urethra is U.S. Pat. No. 5,090,424 granted to Simon et al. for a conformable urethral plug to regulate incontinence.

Besides inflatable cuffs, sleeves and plugs, many other means have been tried to get past the constriction of the external urethral orifice and then achieve desired degrees of deliberate pressure and friction within the urethra. For example, U.S. Pat. No. 5,483,976 granted to McLaughlin et al. discloses a urethral plug that is mechanically actuated by a complex mechanism of connected elements to expand the plug after insertion. The expansion amount is fixed and the design allows no leeway for adjustment of the pressure it intentionally exerts on the urethra other than through careful size pre-selection. Somewhat more forgiving, U.S. Pat. No. 5,131,906 granted to Chen discloses a spherical incontinence device that is longitudinally stretched by an inside plunger during insertion and then resumes its spherical shape upon release, ostensibly regulating distension of the urethra by its pre-selected size and resilient material characteristics, but still relying mainly on pressure and friction. Largely following the same principle, U.S. Pat. No. 2,649,854 granted to Salm discloses an instrument for closing the urethra that also employs a plunger to stretch and reduce the diameter of an elastic element during insertion or removal, its difference being that it is intended to tightly “clamp” the constriction of the orifice between the expanding elastic element and an outside flange when the plunger is released, rather than merely press laterally against the urethral wall.

But at least these last three inventions provide some proactive means for reversing the expansion process. Most devices from the prior art are not so considerate. For example, U.S. Pat. No. 5,513,660, also granted to Simon et al., discloses an expandable urethral plug constructed of shape memory polymer that when activated by body temperature expands from an initial cylindrical shape to a plug that conforms to the dimensions of the urethra with minimal distension. There is no reverse procedure for removal however and the plug must be forcibly pulled out of the urethra and past the constriction of the urethral orifice in its expanded state. Similarly, U.S. Pat. No. 5,671,755, again granted to Simon et al., discloses a disposable foam or elastomer plug for urinary incontinence that is pre-compressed and expands when expelled from an applicator. It too must be forcibly pulled out in the expanded state.

The chief drawback of all these pressure and friction dependent intraurethral devices in the second category is that they seem oblivious to experiences in the real world and the extreme pain and discomfort they can cause. Although it can become somewhat desensitized thru regular use of catheters and such, the urethra is very delicate and easily irritated by even very small and non-engaging implements. Clamping or expansionary devices that deliberately put unrelenting continuous pressure on the urethra, however slight, can become very painful over time and can interfere with circulation and lead to necrosis, abrasion, scarring and other serious long-term consequences. Those that rely on inflation by a pump or syringe, or that are mechanically actuated, typically also have few safeguards and are difficult to adjust to the exacting confines of the urethra, running the risk of immediate injury if accidentally overextended. Those that include friction-increasing serrations or the like can be even more painful and risky. In all cases their reliability and efficacy are also inherently questionable. Inflatable devices can slowly lose pressure and fail. And any device which relies solely on friction, without a physically limiting barrier like the external meatus or bladder wall to act against, will inevitably tend to creep or migrate over time regardless of its design, its effectiveness in occluding the urethra or staying in one place long enough being directly proportional to the detrimental forces it exerts on the urethral wall.

A third broad category in the prior art, and the one that may be considered to include the present invention, involves methods and devices that rely in whole or in part on certain unique physical and anatomical characteristics of the male urethra: namely the aforementioned fossa navicularis and its differentiation from other parts of the urethra. Within the shaft of the penis, the corpus cavernosum urethrae is narrow and of uniform size, measuring about 6 mm. in diameter according to most literature. Proceeding outward from the corpus cavernosum, a pronounced enlargement naturally occurs within the glans penis to form a small chamber known as the fossa navicularis that ends abruptly at the external urethral orifice. The external urethral orifice is the most constricted and unyielding part of the urethra. It is a slit, very short in axial length and narrower than any other part of the urethra, including even the membraneous portion that passes through the sphincter, and is externally bounded on each side by small labia that in combination form the meatus or entrance. The fossa navicularis, besides being larger, is relatively expandable and deformable, able to locally widen by shortening in the longitudinal direction (like a knitted sock), and also able to dilate over time. In the extreme it can accommodate objects more than twice the diameter of the external orifice. In contrast, the short external urethral orifice comprises in part of a narrow ring or band of tough inelastic tissue that is also the terminus of key ligaments. This ring or band of ligament-like tissue, which is embedded just below the meatus and right at the outer end of the fossa navicularis, strongly resists distension and long-term dilation. Wherever reference is made herein to the external urethral orifice, or simply to the urethral orifice or orifice, it is meant to include the meatus and this tough constricting band. These unique characteristics of the male urethra, which are the foundation of the present invention, have in certain instances and to varying degrees also been exploited by the prior art.

Documented applications that rely on the relative inelasticity of the urethral orifice and utilize the enlargement of the fossa navicularis to occlude the urethra and/or hold devices in place exist but are relatively few compared to the other techniques, and leave much to be desired. One of the most functionally dubious is U.S. Pat. No. 6,102,849 granted to Hakac that discloses a non-surgical penile prosthesis, for assisting men with erectile dysfunction. According to the disclosure, its oblong semi-rigid widened tip is specifically designed to be retained inside the fossa navicularis to hold the device in place, but how it does so is crude. The Hakac invention includes a 3-stage “practice device” to progressively dilate the meatus. After a “training” period during which the practice device is repeatedly inserted and withdrawn several times daily until the largest size ostensibly can be comfortably used, the actual prosthesis with its enlarged oblong tip is then inserted. It too dilates the meatus, the assertion by Hakac being that the “dilated meatus very quickly returns to normal size” to keep the inserted prosthesis “securely locked in place during use”. After use, the prosthesis is simply pulled or squeezed out, again necessarily dilating the meatus in the process. Unavoidably relying on this two-way dilation process, it stands to reason that its true comfort and effectiveness could be questioned. Notably, the description fails to explain why the practice device is even needed in the first place. Its intention is obviously to enlarge the urethral orifice, but logic dictates that that would only decrease the size differential between the orifice and the fossa navicularis and reduce the device's retentive effectiveness. What purpose does enlarging the urethral orifice serve?

It is possible that retention of the urinary control device by Salama, mentioned earlier, similarly may depend on the same two-way dilation process. Although it ostensibly relies on friction, the documentation specifically identifies the fossa navicularis as the intended location of the globular valve portion. But it gives no indication how it might be placed there, leaving one to assume it must be small enough to pass through the urethral orifice and hence be of questionable efficacy. Also without any proactive means to aid in its insertion or removal, U.S. Pat. No. 3,463,141 granted to Mozolf discloses a spring-loaded hollow rubber plug that must be forcibly pressed into the urethral orifice to reside both there and within the fossa navicularis for contraception or incontinence purposes.

Improving on the insertion process but not on removal, U.S. Pat. No. 3,373,746 granted to White et al. discloses an internal prophylactic consisting of a spermicide impregnated outer porous plug with an inner adsorption member that together are compressed inside a gelatin capsule for easy insertion, whereupon the capsule ostensibly dissolves and the plug assembly expands inside the fossa navicularis. It is said that the spermicidal jelly will cause the semen to congeal, forming a seal and leading both foam members to expand even further, which casts doubt on its claimed ease of removal by simply pulling an attached string or other external appendage. Also no better from a removal standpoint, U.S. Pat. No. 5,562,599 granted to Beyschlag discloses a urethral damming device to control incontinence that consists of a hollow resilient plug that is open at the distal end and membranous or thin throughout much of its length, but thickened at the proximal end to provide structural rigidity and resistance to expulsion through the constriction of the urethral orifice. A distinction of this invention is that pressure and sealing against the urethral wall is professed to be achieved via fluid pressure of the urine, more so than by the unrelenting bias of the plug material itself. As with the others, the ease and comfort with which it can be removed by simply pulling on a string however remains highly questionable.

All these inventions in the preceding two paragraphs seem to defy the laws of physics and common sense. It is illogical to expect that any device capable of resisting expulsion from the fossa navicularis during ejaculation or micturition, or which requires urethral dilation for insertion, or which requires expansion from a lesser size to hold it there in the first place, can a moment later be extracted with ease and comfort by merely pulling on a string or other appendage without some more active means of reversing the insertion and/or expansion process. One might wonder if such methods have ever actually been tried or are just fanciful speculation and wishful thinking.

Addressing the insertion and removal problem more positively, and also limiting or eliminating any continuous unrelenting pressure on the inside of the fossa navicularis by virtue of their predetermined and relatively fixed dimensions, are the devices disclosed by U.S. Pat. Nos. 4,183,358, 5,701,914 and 5,884,629 granted to Cohen, Loeffler and O'Brien respectively. All three of these inventions are intended for incontinence and/or contraception and all employ reversible mechanical means to allow an elastic element to expand and form a more or less rigid plug inside the fossa navicularis. The O'Brien device is similar to the Chen and Salm devices in that it employs an elastic element that is longitudinally stretched by an inside plunger during insertion or removal, but it leaves unanswered some key design problems that jeopardize its feasibility, like disposition of the volume lost inside the deformable element when the device is stretched. Employing a different technique, the Cohen device is limited by simple geometry in its ability to expand inside the fossa navicularis, i.e. by the dimension of the displacing element that is inserted to force the expandable element laterally outward, because the displacing element must be of a size small enough to leave room for the expandable element where it occupies the urethral orifice. Likewise, the cam-actuated Loeffler device claims an expansion range of only 10 to 14 percent more than its contracted diameter. With limited capacity to expand, both latter devices are restricted in their ability to fully utilize the capacity of the fossa navicularis and to thereby reliably and comfortably prevent expulsion. Analysis indicates that they may instead have a tendency to uncomfortably distend the urethral orifice like a wedge when pressure builds and be prone to unwanted expulsion. Common to all is that they have complex actuating mechanisms that must of necessity be small enough to fit inside the urethral orifice. A methodology of expansion or enlargement that doesn't depend on such size-limited plunger arrangements or other complex mechanisms for operation, such as disclosed herein, would be a lot simpler and more cost effective.

Using a simpler approach, and one that comes closer to the present invention, U.S. Pat. No. 5,603,335 granted to McClenahan discloses an intraurethral contraceptive device that consists of an external container bag for collection of semen which, for sealing engagement with the urethra, is bonded to a thin oblong ring that together with the neck of the bag is inserted edgewise through the urethral orifice and then turned and held in place within the fossa navicularis, somewhat like a button on a shirt. Logically, this method of engaging the fossa navicularis appears to have some merit, but its effectiveness is far from optimum and it has limited other applications because the neck of the bag that is bonded to the retaining ring must be flexible and thin enough to be able to pass through the urethral orifice alongside the ring without reducing the size of the ring to the point that the button-like retentive action becomes inadequate. Yet the neck of the bag must also be sufficiently rigid and strong enough to keep the attached ring correctly oriented and prevent it from being squeezed sideways and potentially expelled or blocking the inlet to the bag. This side-by-side configuration during insertion or removal of the McClenahan invention is seen by this inventor as a serious disadvantage and design constraint. A separate retaining element, whose size is not influenced by any other engaging device during the insertion/removal process and which conversely does not place design limitations on that other engaging device, as disclosed by the present invention, would permit optimizing retentive ability without creating potential orientation problems during use and might be more preferable, especially in other applications where the forces placed on the retaining element may be greater. So too would a retaining element that can remain connected in an end-to end fashion, rather than in a side-by-side fashion, during insertion or removal. Such is also disclosed herein.

Lastly, the discussion would not be complete without also again mentioning inflatable balloons, collars and cuffs. In the context of retention within the fossa navicularis such devices have been used for many years, a good example being for retrograde urethrograms (x-rays with contrast mediums) as improved upon by U.S. Pat. No. 5,713,861 granted to Vanarthos. Besides the drawbacks to pressure and friction devices already mentioned, a disadvantage of such inflatable devices is that the tensile or traction force that can be applied with them, or the expulsive force that they can resist, is often much less than desired. Elastic gas or liquid filled balloons do not hold their shape well and tend to slip through narrow orifices with relatively little resistance other than friction because the internal fluid will usually just flow unrestrained from one side of the constriction to the other while the balloon slides through. Tensile or expulsive limitations notwithstanding, in the case of the male urethral orifice the pressure and friction that this tendency towards fluid transfer and sliding action places on the constriction of the urethral orifice can become quite uncomfortable and painful and limits their usefulness. Even so, given the absence of a better alternative until now, their use may have been considered preferable to more rigid devices that have less margin for safety and increase the chance and severity of injury if excessive force is accidentally applied.

### BRIEF SUMMARY OF THE INVENTION

In summary, a lot of ways have been tried to achieve fixation and/or occlusion of the penis, but none of them seem to be without major drawbacks. It would be ideal if there were an inexpensive and simple way to hold intraurethral devices, like catheters, valves, occluders and prosthetics, unequivocally and securely in place inside the human penis with no chance of expulsion or release, or alternatively that in such use could safely limit and preset the amount of force that can be exerted on the urethra by those devices before expulsion or release occurs; if there were a way of fixating intraurethral devices to the human penis that does not materially influence the size of those devices or put any other significant design constraints on them other than those imposed by the urethra itself; if there were a proactively reversible way of fixating male intraurethral devices that provides simplicity, ease and comfort during both the insertion and removal process without distending or placing significant pressure or friction on the external urethral orifice during either process; if there were a way of fixating male intraurethral devices that does not depend on purposefully applying unrelenting pressure and friction on the urethral wall during use in order to function or maintain position and that would exert minimal stress on the delicate tissues of the urethra to maximize the comfort of the user and minimize adverse bodily reactions; if there were a way of fixating any device, internal or external, to the penis that would allow the application of strong or continuous forces, especially without its performance being influenced by penile changes between the flaccid and erect states; if there were a better way of simply occluding the male urethra that does not have the performance limitations or involve the stress and discomfort of current methods and devices; and lastly if there were a non-penetrative but equally just as secure or positive way of attaching jewelry and other objects or devices to the penis that would not result in scarring or disfigurement and that could be used as an alternative to body piercings for personal adornment or sexual expression and other activities of an erotic nature.

Disclosed herein is an invention that satisfies all of the above ideals and more. It is a novel intraurethral retainer that, alone or in combination with a separate keeper, always has essentially just one primary or key purpose: to cooperatively facilitate fixation of objects or devices to the human penis; other purposes such as for occlusion of the male urethra being perhaps sometimes the ultimate goal but secondary or subordinate in the sense that fixation will technically always be a necessary requisite to such action. With this as its foremost or key purpose, the retainer functions, like some devices in the prior art, by intentionally residing within the fossa navicularis while in use and utilizing the constrictions of the external urethral orifice and the corpus cavernosum urethrae as effective physical barriers to the retainer's dislocation from its position there, but has the distinction of doing so in novel and much more effective ways than anything from the prior art: notably in ways that can virtually preclude any chance of unintentional expulsion or release and that do not of their own accord, in the absence of other forces that might be placed on the retainer, the keeper or the penis itself, put any significant or appreciable pressure or friction on the urethra during the retainer's and keeper's placement and use, despite the fact that the retainer, in order to function, depends on being materially larger than either the external urethral orifice or the corpus cavernosum urethrae while in such use. The words “comfortable”, “significant” and “appreciable” and their derivatives, wherever used herein in reference to the pressure, friction and attendant discomfort that the invention might create, are in this regard intended to refer to the thresholds at which typical users might realistically become reluctant to use the retainer and keeper, those users realizing that some discomfort will be unavoidable and must necessarily be tolerated due to the natural sensitivity of the urethra to the intrusion of any foreign object, but also being aware that they may have other options if it becomes too much.

The retainer and keeper are particularly novel in that they are separate cooperating entities which can be largely generic in nature (like complimentary ordinary nuts & bolts) and not closely tied to any particular class or group of penile devices, or necessarily even to each other. Previously, penile devices that depended on some form of penile fixation always involved physically and inseparably incorporating the means or method for that fixation into the devices themselves, which seriously constrained their design and function. As such, the retainer and keeper have the unique ability to be used in many different applications, individually or in combination, for a broad range of different purposes, their own joint purpose in such use being solely to cooperatively facilitate fixation and/or occlusion of the penis, ancillary to whatever other reasons or objectives may be associated with the devices that employ them, the primary purpose of the retainer, whether for fixation alone or also for occlusion, being merely to hold securely inside the penis whatever engaging device with a keeper or the functional attributes of a keeper that employs it, or by incorporating the attributes of a keeper directly into the retainer, to act in that capacity itself, and the primary purpose of the keeper being merely to engage the retainer in a fixative relationship and facilitate the retainer's use in those cases where a device that incorporates a keeper or that has the functional attributes of a keeper is needed. In that sense the retainer and keeper are indeed very much functionally analogous to ordinary nuts & bolts or threaded studs, with similar flexibility of application and usage, and serve a need that has never been separately addressed before, with all the potential advantages and benefits that that separateness and singularity of purpose entails when incorporating and employing them in those various applications, including the flexibility to readily adapt to varying size requirements and the ability in many cases to interchangeably and selectively mix and match their various embodiments to suit exact needs.

Reduced to basics, the retainer's description boils down to its performance of two key tasks. The retainer's foremost task is to secure its own retention within the fossa navicularis, which it accomplishes by having various novel means to be materially larger than the external urethral orifice or the corpus cavernosum urethrae while it is in its operational state inside the fossa navicularis. In some embodiments, that alone can be enough to allow the secure attachment of external devices to the penis and/or to effectively occlude the urethra. In other embodiments the retainer requires a separate keeper or a device with the functional attributes of a keeper in order to complete its mission and be of any practical use, its task then also being to securely retain that keeper or other device inside the penis, which it has the novel means to accomplish without that keeper or other device needing to be engaged or connected to the retainer until after the retainer itself has been placed in the fossa navicularis. As such, the retainer can be useful on its own, without necessarily even requiring a keeper or any other device to function, and derives its novelty as much from its unique separateness as it does from the improved non-stressful and secure ways retention is achieved.

From a physical and operational standpoint, the detailed descriptions in this disclosure identify three fundamentally distinct embodiments of the retainer, termed herein as its “disk embodiment”, its “cup embodiment” and its “bulb embodiment”. Contrary to the impression these names might give, what differentiates these embodiments is not their implicit shape but rather their material and physical characteristics insofar as those characteristics affect the retainer's manner of insertion or removal, the consequent amount of pressure and friction those processes inherently place on the urethral orifice, and the dependence that the embodiments have on a separate keeper or on a device with the functional attributes of a keeper. They are actually just categorizations of the retainer's many possible embodiments that are given simply to foster a clearer understanding of the invention and that do not limit the scope of the invention in any way. They are useful in conceptualizing and visualizing the retainer in an orderly comprehendible fashion, given that its physical embodiments can be so varied.

Disk embodiments are distinguished by the fact that in their transitional state, i.e. while they are being inserted or removed, they are neither ever larger than the external urethral orifice nor need to be flexible or deformed for insertion or removal and hence can be inserted or removed essentially without any significant pressure or friction on the urethral orifice whatsoever. They can be made of hard unyielding materials like metals if desired and they depend on a separate keeper or other device with the functional attributes of a keeper only to keep them correctly oriented within the fossa navicularis during use (their operational state) and to act as the means for their fixative connection to those devices. The many ways they can accomplish this will become clear in the detailed description that follows.

Cup embodiments on the other hand, regardless of their actual shape, will intrinsically always be somewhat larger than the urethral orifice while in their transitional state, requiring them to be made of deformable materials so that their size and shape may be altered during the insertion or removal process, the consequence of that action being that they will typically exert more pressure and friction on the urethra during that process due to the reactive resilience of the material, however slight that might be, unless specialized applicators or other instruments are employed to mitigate it. Cup embodiments will also depend on a separate keeper or other device with the functional attributes of a keeper to maintain their orientation within the fossa navicularis during use and to act as the means for their fixative connection to those devices, but because they are made of deformable materials, may in some cases also additionally depend on those devices to maintain their operational shape.

Bulb embodiments, depending on their design, can have the characteristics of either disk or cup embodiments with respect to their size relative to the urethral orifice while in their transitional state and how much pressure and friction their insertion or removal places on the urethral orifice, and are primarily distinguished by the fact that they require no separate keeper or other similar device to be functional and useful, because the functional attributes of a keeper are contained in the retainer itself.

The keeper, as claimed herein, is simply a means for enabling use of disk or cup embodiments of the retainer and cooperatively facilitating their operation, with all the physical and functional requirements that that may entail, and serves no other purpose on its own. It derives its novelty and usefulness and its very existence entirely from the retainer. In that respect its description also boils down to the performance of two key tasks: one, to engage the retainer in a fixative relationship, following the retainer's placement within the fossa navicularis, and thereby maintain the retainer's operational state within the fossa navicularis while it is so engaged; and two, by such engagement cooperatively assist whatever device, internal, external or both that incorporates the keeper, in remaining securely fixated to the penis and/or occluding the urethra while it engages the retainer.

Because one of the intentions of the invention is to allow disk and cup embodiments to be used with known devices from the prior art, and because in such use it is conceivable and indeed anticipated that some of those devices will be able to perform like a keeper without any physical modification whatsoever, it is necessary to narrow the definition of the keeper so that it does not read on anything from the prior art. To simply say it is a retainer-enabling device is not enough. Accordingly, the keeper is herein defined as comprising intraurethral elements and operational features of a cooperating device that are not just functionally but also physically identifiable as being specifically retainer-enabling, although retainer-enabling does not necessarily have to be their exclusive purpose, i.e. that enable the device to cooperatively function with a disk or cup embodiment of the retainer for the purpose of intraurethral fixation and/or occlusion, regardless of whether the device is ostensibly intended to be used or is actually used in that cooperative manner, provided those elements and operational features of the cooperating device are physically distinguishable from the prior art insofar as their retainer-enabling characteristics are concerned and their ability to enable use of said retainer is not just the result of mere happenstance or fortunate coincidence. By including the key phrase “not just functionally but also physically identifiable” in this definition, the intention is to clearly differentiate between existing devices from the prior art that by coincidence and without any physical design modification are able to perform the functions of the keeper and make opportunistic use of the retainer and those devices from the prior art that require physical modification to do so, the former being excluded and the latter falling within the scope and definition of the keeper.

As its definition implies, the keeper itself will rarely, if ever, comprise a cooperating device whose elements and operational features cannot also be identified as serving some other additional function or purpose. Usually the keeper will be an integral part of a separate cooperating intraurethral device in which the keeper's function and purpose relate only to permitting the device's use with disk or cup embodiments of the retainer and in which that function and purpose will typically be auxiliary and incidental to other objectives and functions of the cooperating device. Whether or not that cooperating device performs other functions as well is not material to whether it meets the definition of a keeper. In the terminology used herein the word keeper is therefore, for simplicity, intended to interchangeably mean the actual keeper itself or the entire cooperating device that in whole or in part incorporates the elements and operational features that meet the definition of a keeper, there being no meaningful difference between the two insofar as the novelty of the invention is concerned. That is not to say, however, that the cooperating device cannot also be considered novel and new in other ways and an invention in its own right, in addition to being regarded as a keeper or incorporating a keeper.

In light of the possibility that existing devices from the prior art may act as a keeper, it should also be noted that, in the terminology used herein, a distinction is thus always made between a keeper, or other device with the functional attributes of a keeper. The two are deliberately always referred to as separate alternatives (not equivalents) because, as implied, a cooperating device with the functional attributes of a keeper may not necessarily be a keeper or qualify as incorporating a keeper by the precise definition used herein. Several examples of how the keeper's definition applies, and representations of some of the keeper's possible embodiments, are given in the detailed description that follows.

Further technical details, design criteria, functional features, operational objectives and advantages of the entire invention will become apparent from consideration of the drawings, detailed description of the invention and claims that follow. It is to be understood that the particular physical forms, methods of engagement and various applications of the embodiments of the invention that appear herein are given and shown by way of example only and are not to be construed as limitations of the invention. The principles and features of this invention may be employed in various and numerous other embodiments and applications without departing from the scope and concept of the invention.

### BRIEF DESCRIPTION OF THE SEVERAL VIEWS OF THE DRAWING

FIG. 1 is a sectional view corresponding to the plane that describes its least perimeter when viewed in silhouette (that plane also happening to be centrally located and schematically corresponding to Section 1-1 shown in the opposite sectional views of FIGS. 5, 6 & 7) of a representative one-piece disk embodiment having a simple flat shape with round edge in that cross-section.

FIG. 2 is a sectional view corresponding to the plane that describes its least perimeter when viewed in silhouette (that plane also happening to be centrally located and both schematically and dimensionally corresponding to Section 2-2 shown in the opposite sectional views of FIGS. 5, 6 & 7) of a representative one-piece disk embodiment having a more semi-elliptic shape in that cross-section.

FIG. 3 is a sectional view corresponding to two coincident planes describing the least perimeters of its two elements when each is viewed in silhouette (each plane also happening to be centrally located and schematically corresponding to Section 3-3 shown in the opposite sectional views of FIGS. 5, 6 & 7) of a representative two-piece disk embodiment also having a symmetric semi-elliptic shape in that cross-section, but only when the two elements are used in combination.

FIG. 4 is a sectional view corresponding to four coincident planes describing the least perimeters of its four elements when each is viewed in silhouette (each plane also happening to be centrally located and the views of the two inner elements schematically corresponding to Section 4-4 shown in the opposite sectional views of FIGS. 5, 6 & 7) of a representative four-part disk embodiment that in combination forms a spherical shape in that cross-section.

FIG. 5 is a sectional view through the plane of maximum outside perimeter, schematically corresponding to Section 5-5 shown in the opposite sectional views of FIGS. 1, 2, 3 & 4 and dimensionally consistent with FIG. 2, of a representative disk embodiment that is of basic size (not extra-sized), whose cross-sectional area in that plane is hence minimal and round in shape for that nominal size group. With the exception of the sectional references, FIG. 5 is also representative of a plan view of the cup embodiment shown in FIG. 8.

FIG. 6 is a sectional view through the plane of maximum outside perimeter, schematically corresponding to Section 6-6 shown in the opposite sectional views of FIGS. 1, 2, 3 & 4 and dimensionally consistent with FIG. 2, of a representative disk embodiment that is in the same nominal size group as that of FIG. 5 and that has the same width W, but is elongated by 2 mm in the L dimension (definitively “extra-sized”) to increase the cross-sectional area in that plane and hence its retentive abilities, making it slightly elliptic in shape but still able to pass through the same size orifice. With the exception of the sectional references and the dimension L, FIG. 6 is also representative of a plan view of the cup embodiment shown in FIG. 8.

FIG. 7 is a sectional view through the plane of maximum outside perimeter, schematically corresponding to Section 7-7 shown in the opposite sectional views of FIGS. 1, 2, 3 & 4 and dimensionally consistent with FIG. 2, of a representative disk embodiment that is also in the same nominal size group as those of FIGS. 5 & 6 and also has the same width W, but is elongated by 4 mm in the L dimension (definitively more “extra-sized” than in FIG. 6), further increasing the cross-sectional area in that plane and hence its retentive abilities and making it even more elliptic in shape, but also still able to pass through the same size orifice. With the exception of the sectional references and the dimension L, FIG. 7 is also representative of a plan view of the cup embodiment shown in FIG. 8.

FIG. 8 is a sectional view through a plane coincident with its axis, of a representative example of a basic cup embodiment of the retainer in its uncompressed or operative state.

FIG. 9 is a perspective view, normal to the direction of insertion, of a representative example of a cup embodiment of the retainer similar to that of FIG. 8 that has been compressed along a plane coincident with its axis in preparation for insertion, depicting the method of insertion. This figure also essentially applies to the method of inserting certain bulb embodiments.

FIG. 10 is a perspective view, facing the direction of insertion, of a representative example of a cup embodiment of the retainer similar to that of FIG. 8, or of a representative example of a bulb embodiment of the retainer similar to that of FIG. 11, that has been compressed along a plane coincident with its axis in preparation for insertion, showing the critical size in that state.

FIG. 11 is a sectional view through a plane coincident with its axis, of a representative example of a bulb embodiment of the retainer in one of its most basic forms: uniform in wall thickness and devoid of internal or external grooves or ridges.

FIG. 12 is a sectional view through a plane coincident with its axis, of a representative example of a basic bulb embodiment of the retainer that incorporates a separate passage through the retainer for the drainage or introduction of fluids.

FIG. 13 is a sectional view of a human penis illustrating use of a bulb embodiment of the retainer.

FIG. 14 is a sectional view of a human penis illustrating novel use of the invention in cooperation with a short spout that has all the definitive elements of a keeper, to allow the attachment of jewelry (a captive-bead ring in this case) and/or to emulate the appearance and function of a Prince Albert piercing, among numerous other uses.

FIG. 15 is a sectional view of a human penis illustrating use of a disk embodiment of the retainer in cooperation with an ordinary rubber catheter that has been adapted to its use by the addition of two small raised annular ribs on its surface, those ribs definitively qualifying the catheter as incorporating a keeper.

FIG. 16 is a sectional view of a human penis illustrating use of a cup embodiment of the retainer in cooperation with a urinary control valve similar to that disclosed by Salama, as referenced in the background herein, the valve in this case requiring no physical modification and hence not qualifying as incorporating as a keeper but merely as a device with the functional attributes of a keeper.

### DETAILED DESCRIPTION OF THE INVENTION

The invention comprises a novel intraurethral retainer and keeper. More precisely, the invention comprises an intraurethral retainer that, alone or in cooperation with another device that incorporates a compatible keeper or that has the functional attributes of a compatible keeper, has essentially just one primary or key purpose: to facilitate fixation of objects or devices to the human penis; other purposes such as for occlusion of the male urethra being perhaps sometimes the ultimate goal but secondary or subordinate in the sense that fixation will technically always be a necessary requisite to such action. It is a device that by itself can used in lieu of some of the other methods and devices mentioned in the background herein as a possibly much better alternative, but most notably it is a separate device that can be cooperatively adapted and used to securely retain within the penis essentially any other device that is small enough to be inserted into the penis without itself materially influencing the size or design of that other device, the only constraint being that the other device incorporates a compatible keeper or has the functional attributes of a compatible keeper.

With this singularity of primary or key purpose, the retainer and its cooperating keeper function like some devices in the prior art, by intentionally residing within the fossa navicularis while in use and utilizing the constrictions of the external urethral orifice and the corpus cavernosum urethrae as effective physical barriers to the retainer's and keeper's dislocation from their position there, but that have the distinction of doing so in novel and much more effective ways than anything from the prior art: notably in ways that can virtually preclude any chance of unintentional expulsion or release and that do not of their own accord, in the absence of other forces that might be placed on the retainer, the keeper or the penis itself, put any significant or appreciable pressure or friction on the urethra during the retainer's and keeper's placement and use, despite the fact that the retainer, in order to function, depends on being materially larger than either the external urethral orifice or the corpus cavernosum urethrae while in such use. The words “comfortable”, “significant” and “appreciable” and their derivatives, wherever used herein in reference to the pressure, friction and attendant discomfort that the invention might create, are in this regard intended to refer to the thresholds above which typical users might realistically become reluctant to use the retainer and keeper, those users realizing that some discomfort will be unavoidable and must necessarily be tolerated due to the natural sensitivity of the urethra to the intrusion of any foreign object, but also being aware that they may have other options if it becomes too much.

The retainer and keeper are particularly novel in that they are separate cooperating entities which can be largely generic in nature (like complimentary ordinary nuts & bolts) and not closely tied to any particular class or group of penile devices, or necessarily even to each other. Previously, penile devices that depended on penile fixation always involved physically and inseparably incorporating the means or method for that fixation into the devices themselves, which seriously constrained the devices' designs and their function. As such, the retainer and keeper have the unique ability to be used in a broad range of different applications, individually or in combination, for a multitude of purposes, their own joint purpose in such use being solely to cooperatively facilitate fixation and/or occlusion of the penis, ancillary to whatever other reasons or objectives may be associated with the devices that employ them, the primary purpose of the retainer, whether for fixation alone or also for occlusion, being merely to hold securely inside the penis whatever engaging device with a keeper or the functional attributes of a keeper that employs it, or by incorporating the attributes of a keeper directly into the retainer, to act in that capacity itself, and the primary purpose of the keeper being merely to engage the retainer in a fixative relationship and facilitate the retainer's use in those cases where a device that incorporates a keeper or that has the functional attributes of a keeper is needed. In that sense the retainer and keeper are indeed very functionally analogous to ordinary nuts and the bolts or studs that fix onto them, with similar flexibility of application and usage, and serve a need that has never been separately addressed before, with all the potential advantages and benefits that that separateness and singularity of purpose entails when incorporating and employing them in those various applications, including the flexibility to readily adapt to varying size requirements and the ability in many cases to interchangeably and selectively mix and match their various embodiments to suit exact needs.

To be precise, the retainer performs two key tasks: one, to effect its own retention within the fossa navicularis and thereby achieve fixation to the human penis, whether or not that also occludes the urethra; and two, to thereby fixate to the penis any device that cooperatively engages it, whether that device is internal, external or both. The retainer's novelty, aside from the non-stressful and secure way it accomplishes its objectives, as will be further detailed herein, is that it is a separate device which in certain embodiments requires it to be intraurethrally engaged by a keeper or by a device with the functional attributes of a keeper in order to make it functional and operationally useful, but that notably does not require it to be attached to or physically part of any such cooperating device during the insertion or removal process, making the retainer a unique stand-alone device that can be independently useful with a variety of engaging devices (not just specific keepers) having a variety of different purposes.

In that cooperative context, the keeper gets its novelty from the retainer. The keeper is simply a means for enabling use of certain embodiments of the retainer and facilitating their operation in certain applications, with all the physical and functional requirements that that may entail, and serves no other purpose on its own. It derives its novelty and usefulness entirely from the retainer and owes its very existence to the retainer. In that respect it also performs two key tasks: one, to engage the retainer in a fixative relationship, following the retainer's placement within the fossa navicularis, and thereby maintain the retainer's operational state within the fossa navicularis while it is so engaged; and two, by such engagement cooperatively enable whatever device, internal, external or both that incorporates the keeper, to remain securely fixated to the penis and/or occlude the urethra while it engages the retainer.

As mentioned in the summary, the retainer can have myriad physical forms. Operationally and functionally these can be grouped into three distinct embodiments, referred to herein as its “disk embodiment”, its “cup embodiment” and its “bulb embodiment”. Contrary to the impression these names might give, what differentiates these embodiments is not their implicit shape but rather their material and physical characteristics insofar as those characteristics affect the retainer's manner of insertion or removal, the consequent amount of pressure and friction those processes inherently place on the urethral orifice, and the dependence that the embodiments have on a separate keeper or on a device with the functional attributes of a keeper. They are actually just categorizations of the retainer's many possible embodiments that are given simply to foster a clearer understanding of the invention and that do not limit the scope of the invention in any way. They are useful in conceptualizing and visualizing the retainer in an orderly comprehendible fashion, given that its physical embodiments can be so varied.

Disk embodiments and cup embodiments are the most novel and unique. They are distinguished from the prior art by their unique separateness and specialized primary purpose and by the fact that they are designed for use only in cooperation with a keeper, or with a device that has the functional attributes of a keeper, and indeed depend on that cooperation in order to function. In a sentence, either of these two embodiments can therefore be described simply as an intraurethral retainer, for use with a cooperating device that incorporates a compatible keeper or that has the functional attributes of a compatible keeper, to facilitate fixation and/or occlusion of the human penis in joint use with that cooperating device, that action being its primary or foremost purpose, the intraurethral retainer comprising a separate device that is able to be placed within the fossa navicularis of a human penis separately from the cooperating device, and that, while it is operationally positioned within the fossa navicularis and while it is engaged by the cooperating device, is able to prevent or impede its own dislocation and correspondingly retain the cooperating device within the penis by utilizing the comparative (relative to its own size when operationally positioned within the fossa navicularis) constrictions of the urethral orifice and the corpus cavernosum urethrae as effective physical barriers to the intraurethral retainer's dislocation from its position.

But it is not just their separateness that makes disk and cup embodiments novel and unique. It is also the fact that they neither cause nor necessitate dilation or distension of the urethral orifice or exert significant pressure or friction on the orifice during placement. Nor do they of their own accord and in the absence of any other force exerted on the retainer, the cooperating device or the penis, exert significant pressure or friction on the urethral wall while in place. These are not merely advantages, but specifically novel features that set them apart. As a group, if the above sentence is not already descriptive enough, disk embodiments and cup embodiments can therefore also be more precisely but still broadly described by means-plus-function language as also comprising:
first means for sufficiently reducing the perceived French perimeter of the retainer, preparatory to the retainer's insertion into the fossa navicularis or its removal from the fossa navicularis and relative to the retainer's silhouette when viewed from a perspective that is in line with the urethra, such that while its perceived French perimeter from that perspective is so reduced, which is hereinafter referred to as the retainer's transitional state, passage of the retainer through the constriction of the external urethral orifice does not cause or necessitate dilation or distension of said orifice or exert significant pressure or friction on said orifice during such passage;
second means for sufficiently increasing the perceived French perimeter of the retainer, while it is positioned within the fossa navicularis and relative to the retainer's silhouette when viewed from a perspective that is in line with the urethra, such that while so positioned and while its perceived French perimeter from that perspective is so increased, which are hereinafter collectively referred to as the retainer's operational state, the comparative constrictions of the opposing external urethra orifice or the corpus cavernosum urethrae are small enough to function as effective physical barriers to the retainer's passage through them and obstruct the retainer's unintentional dislocation from its position within the fossa navicularis, and also such that, while the retainer is in its operational state, it is simultaneously ensured that the retainer's perceived French perimeter does not exceed that of the fossa navicularis and is still small enough for the retainer to comfortably reside within the fossa navicularis without, of its own accord and in the absence of any other force exerted on the retainer or the penis, exerting significant pressure or friction on the urethral wall; and
third means for the at-will engagement and disengagement of the retainer by the cooperating device, while the retainer is in its operational state, such that when the retainer is engaged a fixative relationship with the cooperating device exists that is sufficient both to maintain the retainer's operational state as well as to share in some measure with the cooperating device the retainer's retentive abilities to remain fixated within the fossa navicularis, and also such that when the retainer is disengaged, no fixative relationship with the cooperating device exists to prevent or obstruct the retainer's return back to its transitional state, at-will meaning entirely under the user's control.

Getting more specific, in the disk embodiment of the invention the retainer comprises one or more rigid or semi-rigid disk-like components of smooth low-friction exterior, that are sized and shaped such that, when each is inserted into the urethra in an edgewise direction normal to the plane of its least French perimeter when viewed in silhouette, the retainer or each of its individual components is able to pass through the external urethral orifice without causing distension of the orifice or exerting appreciable pressure or friction thereon and, when subsequently turned 90 degrees to that plane inside the fossa navicularis, presents facing surfaces that are substantially larger in French perimeter than those of the adjoining external urethral orifice and the corpus cavernosum urethrae, but still small enough to comfortably reside in the fossa navicularis without itself exerting significant pressure or friction thereon in the absence of any other force applied to the retainer or penis.

As a side-note: the term “French perimeter” is herein used to describe the least length of the inside boundary of an opening or lumen, or for outside measurements, the least length of the path that a string or elastic band would take if tightly wound around an object, i.e. in that case coinciding with any convex boundaries and spanning any concave boundaries in a straight line.

After placement within the fossa navicularis, the retainer or each of its components is engaged by the keeper or whatever other device with the functional attributes of a keeper that it is intended to retain, such that singularly or as the assembly of its components, whichever the case may be, it maintains its assemblage and orientation within the fossa navicularis and resists or virtually precludes expulsion or dislocation by any force exerted on the engaging device or the retainer itself that is not intended to cause or result in such expulsion or dislocation, the mode of retention being analogous to that of a common button where the external urethral orifice or the corpus cavernosum urethrae functions as the button hole. Insertion or removal of disk embodiments can be achieved without aid of applicators or other instruments (although this does not preclude using such aids) by simply manipulating the penis externally to squeeze the retainer in or out, independently of any engaging device, relying on the engaging device only to keep the retainer correctly oriented during use and its assemblage (if it has more than one component) intact and possibly, if it is made of resilient materials, to keep it from deforming during use.

The primary distinction of the retainer in its disk embodiment, contrary to its cup embodiment, is that insertion and fit are not dependent on deforming or altering the shape of the retainer itself during the insertion or removal process, although this does not preclude making the retainer of resilient or deformable materials for other reasons or to further aid in the insertion/removal process. Depending on the particular functional objectives and other factors of each case, in its disk embodiment the retainer therefore can be made from virtually any biocompatible material with the appropriate physical and mechanical properties, including metals and their alloys in bare or plated form, as well as other natural and synthetic materials, using ordinary manufacturing techniques. Where cost is not a significant factor, precious or difficult-to-work metals like gold, stainless steel or titanium may be popular for their durability and aesthetic or vanity appeal. In other cases, plastic resins and other synthetic compounds, which can be molded and are often more economical, may be preferred. Flexible or resilient materials like certain synthetic latexes, silicones, polyurethanes and nitrile rubbers, that can be formulated to deform and release the engaging device or to permit the expulsion of the retainer itself while still engaged when enough force is applied, will be especially desirable in some cases for designed safety. They may also be valued for their comfortable shape-conforming characteristics and sealing abilities. Given man's propensity for the unusual, even natural materials like carved stone, gems or mother-of-pearl are not out of the question. In short, disk embodiments are not limited to any particular type or class of material other than those required to satisfy the desired physical and mechanical properties, which will become self-evident herein.

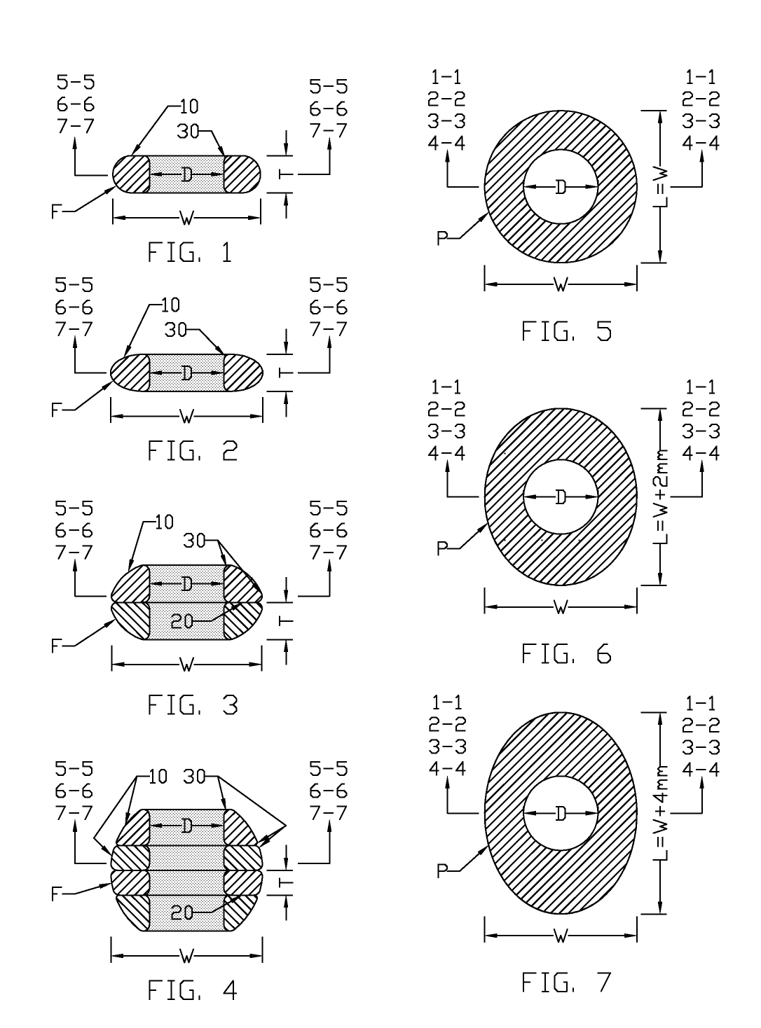

For comparison and clarity of understanding, FIGS. 1 thru 7 all proportionately depict size 26Fr disk embodiments with size 18Fr holes, and the illustrations of FIGS. 5, 6 & 7 dimensionally correspond to that of FIG. 2. Referring to FIGS. 1, 2, 3, & 4, which illustrate sectional views corresponding to the retainers' planes of least perimeter when viewed in silhouette and which also correspond to the sections described in FIGS. 5, 6, & 7, in its disk embodiment the retainer or each of its separate components can take a variety of shapes or forms, each characterized as a disk-like object of functionally, if not actually, solid construction having smooth proximal surfaces 10 relative to where it contacts the urethra, sometimes one or two distal surfaces 20 (depending on whether it is part of a two or more piece configuration) that normally only contact each other when in place, a maximum thickness “T” between its two largest surfaces, a minimum outside width “W” when viewed in silhouette that will correspond to its diameter only if its planar shape is round, and rounded edge(s) 30 wherever any surfaces meet and are not otherwise tangential. In these examples round and elliptical shapes are shown. Although unlikely to be of much benefit, shapes that are neither round or elliptical, or even symmetrical, when seen in plan views like those of FIGS. 5, 6, 7 & 8, are possible. Hence the descriptions of FIGS. 1, 2, 3 & 4 refer to their minimum cross-sections in silhouette, which for these examples are actually the same as their cross-sections on their central planes of minimum girth. The dimension “F” (its French size) describes this minimum outside perimeter or girth (of the largest single component when there is more than one) on this central plane of cross-section corresponding to its minimum width W and for all practical purposes denotes the retainer's critical size, i.e. the minimum size of urethral orifice it can pass through without causing or requiring distension. The dimension P, on the other hand, describes its largest outside perimeter. In cases where the retainer is made of a resilient compressible material, P will remain essentially constant for any retainer whereas the dimensions F and W will not necessarily be fixed and may vary significantly between a maximum uncompressed state and a minimum compressed state. In FIGS. 1 to 7, rigid or uncompressed materials are depicted. In the figures both F and P refer to the French perimeter of the outside boundary that is indicated by the arrow. Typically, although not necessarily, the retainer or each of its components will also be characterized by a central hole or bore of diameter “D” extending perpendicularly to tangential planes between its two major surfaces, the hole also having rounded edges 30. To aid the viewer in understanding the figures, numerals are used to denote features and upper case letters are used to denote dimensions.

For effective use of disk embodiments, the retainer or its largest component should preferably be sized to be just small enough to comfortably pass through the external urethral orifice and usually no smaller. To accommodate typical variations in the human anatomy, and to facilitate easy accurate fitting, the invention thus relies on being manufactured in a range of common conventional sizes: those that are consistent with the French or Charriere system of sizing being the most logical but not necessarily the only system. For the benefit of anyone not already familiar with such sizing systems, common medical appliances like catheters, sounds, valves, stents and other implements intended for insertion in bodily lumen or incisions are nowadays almost universally standardized according to nominal French or Charriere sizes, where the stated size is an approximation of the peripheral circumference C in millimeters of an equivalent round object of diameter D based on the familiar formula for a circle C=πD, but where the constant π (pi) is represented by the whole number 3 rather than a more precise value. Stated another way: French or Charriere size, hereinafter referred to simply as French size, is defined as 3 times the diameter in millimeters of an equivalent round object of equal girth. Hence a size 18Fr catheter or other appliance will have an equivalent diameter of exactly 6 mm but a circumference or girth that is not 18 mm but actually closer to 18.85 mm.

The French or Charriere system of sizing has become commonplace because the incisions, openings and cross-sectional areas of objects that it is applied to may be irregularly shaped and need not actually be round. In the case of trough-like objects that are C or S-shaped in cross-section, it is understood by definition that the measurement applies to the path that a string or elastic band would describe if wound tightly around the object, coinciding with any convex boundaries and spanning any concave boundaries in a straight line. By applying this system of sizing, or others like it, to the minimum perimeter of the retainer or its largest component when viewed in silhouette, i.e. to the dimension F in its various disk embodiments, the measurement will already be familiar to many users and the only thing needed to make an informed size selection, regardless of the particular model or style of retainer being contemplated, will be to determine the French size of the largest object that can be comfortably inserted into the penis without necessitating distension, which can be easily determined by trial and error using ordinary catheters, sounds or other measurable probes and which will likely already be known to many users. However, whether or not any sizing system is used is not intrinsic to the invention and the French system is only used herein to aid in understanding the invention and as a suggested means for facilitating its use.

The holes of diameter D that are shown in FIGS. 1 thru 7 are intended in these examples to act as the means for engaging/disengaging keepers or other cooperating devices. One of the unique advantages and features of the retainer in its disk embodiment (as well as in its cup embodiment) is that when such holes are provided as the means for engagement, those holes can be as large as the urethral orifice that the retainer is intended to pass through and no restriction whatsoever is placed on the size of any engaging device that can be used therewith other than the limiting size of the urethral orifice itself. To facilitate adaptation to existing catheters and valves and some of the other devices mentioned in the background, the invention's usefulness thus also relies on the retainers in some cases being manufactured with varying sized holes in a range of standard sizes for each nominal size of retainer. In such cases the same French sizing system can also apply to the bore such that the designation 26-18 might refer to a retainer that is able to pass through a size 26Fr urethral orifice and that has a size 18Fr hole. It is assumed that increasing the bore slightly to permit a loose fit with standardized existing appliances, or decreasing it slightly to permit a tight fit, would not be considered a departure from this convention and that the norms of established and accepted tolerances in the industry would apply. Additionally, when the retainer has a hole and is made of a resilient material that would allow compression and reduction of the W dimension, the same sizing convention can still apply. Extending the above example, a designation of 26/18−18 would logically imply a flexible 26Fr retainer that, if needed, can be compressed to the same outer size as its hole. Although probably of limited practical value, disk embodiments that can be compressed to even less than the size of their holes are not inconceivable. Even with standardization, an enormous array of different size combinations is thus possible. Using the above format and convention, or one like it, to specify the correct retainer for any particular application should however remain easy and not result in confusion. The invention is, as such, not constrained by any size limitations.

Prototype experiments have shown that in their disk embodiment, retainers that are simply round in plan view may not take full advantage of the capacity of the fossa navicularis. It is therefore an intrinsic feature of the invention that such retainers may, in each nominal size, also be produced in a series of incrementally larger shapes, referred to herein as “extra-sizing”, commencing with the minimum round shape when viewed in plan view and progressively becoming elongated and more elliptical or oblong in shape by incrementally increasing the L dimension, and hence also P, while keeping the width W constant and hence also keeping the minimum cross-sectional perimeter F (the critical nominal French size) constant as depicted in FIGS. 5, 6 & 7. The purpose of such extra-sizing will be to allow users, through trial and error or by measurement, to select a larger retainer that, if desired, more fully occupies the fossa navicularis to optimize retentive ability and comfort. Not essential to the invention, but to increase user understanding and avoid confusion, it is suggested that in these cases the sizing convention could be carried one step further so that, for example, the size designation 26-18+0 might denote a size 26Fr retainer that is round with an 18Fr bore like that in FIG. 5 while 26-18+2 might denote the same nominally sized retainer extra-sized by 2 mm like that in FIG. 6, i.e. 2 mm longer than it is wide when viewed in plan view. Alternatively a convention can be adopted whereby all three perimeters (F, D & P) are simply stated as their French size equivalents. For the round example of FIG. 5 this would be designated as a French size 26-18−36.8 while elliptically extra-sizing by 2 mm as shown in FIG. 6 would result in a French size designation of 26-18−39.8. This might be more meaningful and practical to some users, given that the extra-sizing can be of any shape just as long as the width W (and hence F) does not increase, but doesn't readily indicate that the retainer is extra-sized. Which sizing convention, if any, is adopted may ultimately depend on the retainer's intended use. For demanding applications where comfort and hence precise fit are essential, prototype experiments have indicated that extra-sizing in French size increments of as little as 1 unit may be desirable, which may favor an approach like the second alternative. In any event, adherence to close manufacturing tolerances will likely be important in many applications because any variation in outside width or length will have more than a three times larger effect on both F and P.

The foregoing comments show just some of the many considerations that may come into play when designing and marketing disk embodiments. In that regard and as a general note applicable to all embodiments, it is to be understood that the shapes and configurations illustrated in the figures and discussed herein are given only to foster a better understanding of typical design considerations and functional parameters and are not to be interpreted as constraints on the invention. Virtually any functional shape and configuration may do, the possible combinations being almost limitless, but depending on the materials used and other factors such as cost or comfort, some shapes and configurations may in certain instances be preferable to others. This applies especially to the disk embodiment of the invention.

Generally, for any particular size F, i.e. for any disk embodiment just able to pass through a urethral orifice of comparable size, retentive effectiveness will be optimized by selecting the shape and configuration to make the dimension W as wide as possible. The wider it is, the less extra-sizing will be needed to capitalize on the comparatively larger size of the fossa navicularis and correspondingly the more round and hence more comfortable it will be, especially when push or pull forces are exerted on the retainer by the engaging device. Generally, in anticipation of such push or pull forces, optimizing the width of the retainer so that it butts up against the urethral orifice with the largest and roundest area possible, to emulate the action of an ordinary button and minimize any tendency for it to distend the orifice and push or pull thru, may seem like the most important criteria. But how this affects its cross-sectional shape in regard to other comfort concerns will also usually be just as important. Coupled with the desirability of size standardization, which will also require careful adherence to exacting dimensional tolerances, design trade-offs to achieve a balance between comfort and retentive effectiveness while also taking into account manufacturing costs and quality control, will likely be inevitable, as might be with any product of this sort.

For ease of comparison, the examples of disk embodiments shown in FIGS. 1, 2, 3 & 4, proportionately and regardless of the scale of the drawing, all depict retainers of 26Fr size with an 18Fr size hole and an arbitrary 3 mm thickness T, except FIG. 4 where the thickness T of the two inner components is proportionately 2 mm. These illustrations show that within practical limits of thickness T, the minimum width W will generally not be strongly influenced by shape. To wit, in the examples shown the minimum width W only varies from 11.90 mm (FIG. 1) to 12.26 mm (FIG. 2) which, as the figures demonstrate, is visually an almost imperceptible difference. Nevertheless, prototype tests have shown that even such seemingly minor differences can profoundly affect comfort and effectiveness. This becomes more apparent when one considers their P dimensions, which vary from 35.7Fr to 36.8Fr, a difference of more than 1 unit.

Compressible variations notwithstanding, simple flat one-piece retainers with a round edge, such as shown in FIG. 1, will have the advantage of generally being the easiest to make, but provide the least width W for any given nominal size of retainer. Form-wise they also do not conform well to the naturally domed shape of the fossa navicularis next to the urethral orifice. These two factors make them potentially the least comfortable when strong external push or pull forces are applied via the engaging device, but this may not always be an important consideration or might be mitigated by using flexible resilient materials and their comparatively blunt peripheries could be a significant offsetting factor with regard to any external pressures that the penis might be placed under.

By comparison, a more semi-elliptic one-piece retainer, such as shown in FIG. 2, may be more difficult to make (especially if it requires machining or grinding), but will provide greater width W for any nominal size and conform much better to the shape of the urethra near the orifice. Prototype tests have shown that this seemingly innocuous change in shape can make a profound difference in comfort when strong external push or pull forces are applied via the engaging device. However the relatively sharper outer periphery of such one-piece semi-elliptic designs may be undesirable in cases where external pressure is constantly placed on the penis by the wearer's clothing or other conditions.

At the sacrifice of only a slight decrease in width, a two-part semi-elliptic retainer such as shown in FIG. 3 can provide an even better fit to the inner domed shape of the fossa navicularis near the urethral orifice under push or pull forces transmitted by the engaging device, as well as dramatically increase comfort in cases where the penis is squeezed by the user's clothing or other conditions. Such configurations may also be popular in some instances because either of the two components can be used alone if desired, but production costs, although very modest, are essentially doubled and the process of insertion or removal when used in tandem becomes more complicated.

For maximum comfort, both when strong push or pull forces are transmitted by the engaging device and when the penis is squeezed by the wearer's clothing or other conditions, early prototype tests have shown that a multi-part retainer which is spherical or ellipsoid in shape, such as the 4-part configuration shown in FIG. 4, is best. But comfort under such conditions may not always be an overriding factor (especially once the fossa navicularis becomes desensitized thru regular use) and their complexity of sequential insertion and even higher costs, albeit still very modest, may be more important considerations.

In short, there is no clear-cut preferred shape or configuration, or any indication that there ever will be. Preference will depend on user experience and the circumstances of each case, as well as market forces and myriad other variables. By the same token, the invention is not defined or constrained by any physical shape, material or configuration, but rather by the basic principles whereby it functions and by the concepts of how it is inserted/removed, which together will govern its design, and the illustrations and physical descriptions contained herein are only provided as rudimentary examples to foster a greater understanding of the invention. This same comment applies to all other embodiments as well.

Notwithstanding possible designs made of resilient materials that may allow a degree of deformity due to the presence of a hole or just the material characteristics themselves, a distinction of the disk embodiment of the retainer, compared to the other two embodiments, is that comfortable insertion and fit can be a matter of just selecting the right fixed dimensions and shape and need not involve deforming or altering the shape of the retainer itself during the insertion or removal process. Assuming it is properly sized, this assures the least possible stress on the delicate tissues of the urethra during the insertion or removal process, but at the same time limits the sizes and shapes of the retainers that can be used. There are likely to be many applications where a one-piece retainer that more closely approximates the natural domed shape of the fossa navicularis near the orifice, and/or where a larger size than is possible with a disk embodiment without resorting to oblong extra-sizing, is desirable. In such cases the cup embodiment of the retainer may be more appropriate.

In the cup embodiment of the invention, which is different in form and method of implementation but employs the same fundamental principles of operation, the retainer will typically be a hollow semispherical, parabolic or conical cup-like structure (although flat disk-like structures are not excluded) composed of a springy resilient material that is sized and shaped so that it can be easily compressed along its axis between the thumb and finger of one hand and inserted into the urethra in a direction coincident with the plane of compression and 90 degrees to that axis as depicted in FIG. 9, using a rolling motion of the thumb and finger to squeeze it past the urethral orifice, that action unavoidably exerting some pressure and friction on the orifice but not enough to cause distension or significant discomfort due to the retainer's sufficiently small size and low resilience. Upon entering the fossa navicularis the compressed cup-like retainer is allowed to rebound to its original shape and by external manipulation is then turned 90 degrees with the apex of the cup pointed toward the external urethral orifice and is then engaged by a keeper or other cooperating device, thus presenting a facing area that, like its disk counterpart, is substantially larger in silhouette than that of the urethral orifice or the corpus cavernosum urethrae, but again also still small enough to comfortably reside in the fossa navicularis without itself then exerting significant pressure or friction thereon, and such that it then resists or totally precludes expulsion or dislocation by any force placed on the engaging device or the retainer itself that is not intended to cause or result in such expulsion or dislocation.

Like with its disk counterpart, one of the cup embodiment's key distinctions is that it is a separate device that is not attached to or part of any other device during the insertion or removal process and that can, as such, be used with a variety of different cooperating devices, both existing and novel, having various different purposes, without itself influencing the size of those engaging devices. More so than with disk embodiments, the separate keeper or other engaging device will function not only to keep the retainer correctly oriented during use, but often also to maintain the retainer's shape once it is in place and may be deliberately designed or selected to aid in resistance to shape deformation of the retainer and/or to safely release from the retainer, or to allow expulsion or release of the entire assembly from the penis, when a certain maximum design load is exceeded.

Generally, one of the most desirable objectives in designing cup embodiments will be to allow the retainer to be easily inserted or removed without aid of any applicators or other instruments as depicted in FIG. 9, although this does not preclude using such instruments to further assist in the insertion or removal process. For insertion this might be a thin holder or clamp that keeps the retainer compressed until it enters the fossa navicularis. Removal will generally be accomplished in reverse order, by disengaging the keeper or other cooperating device and then turning and squeezing the retainer back out. Here too it may be more expedient, convenient or comfortable to grab the retainer by a leading edge and pull it out with something like thin forceps, rather than to squeeze it out. Unless a tool is devised that can keep the retainer compressed during both the insertion and removal process without seriously compromising other design considerations like maximum size, the thing to keep in mind is that those actions of insertion or removal will unavoidably exert some pressure and friction on the urethral orifice due to the resilient reaction to deformation of the material as the retainer passes through the constriction. Selecting materials and dimensions to keep that resilient reaction to deformation as light as possible therefore usually will be one of the key concerns during design.

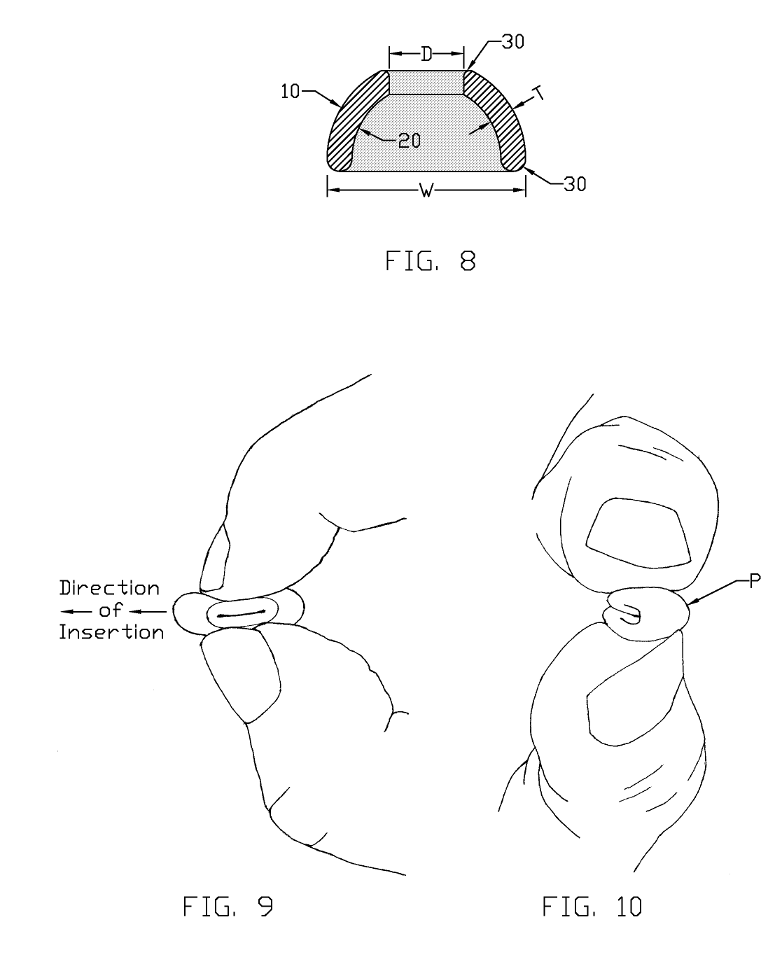

Referring to FIG. 8 which illustrates a sectional view through a central plane coincident with its axis, in its typical cup embodiment the retainer can be characterized as a conical, parabolic or semi-spherical cup-like object (a semi-spherical design is shown) of either solid or hollow construction having a smooth low-friction proximal or outer surface 10, a distal or inner surface 20 and a rounded edge or rim 30 where the outer and inner surfaces converge or meet, with a thickness “T” between the inner and outer surfaces that may either be uniform as shown or may vary to regulate its deformability and the reactive pressure it exerts on the urethral wall under applied load. Typically, although not necessarily, the retainer will also be characterized by a hole or bore of diameter “D” centered on its axis and extending from the outer surface to the inner surface, the hole also having a rounded edge 30 at the outer surface, that hole being the means for engaging keepers or other cooperating devices. By disregarding the sectional references, FIG. 5 can serve a dual purpose and also be interpreted as how this retainer could appear in plan view, although the dimension “W”, which refers to its width, will in this case have no practical significance because it can be so easily distorted and thus only serves to tie the two views together. Indeed, by disregarding the sectional references, FIGS. 6 & 7 also can be equally interpreted as plan views of the cup embodiment shown in FIG. 8, the main dimension of significance in these views being its perimeter P, which will be important to its fit and function when it is in place.

FIG. 8 depicts a typical solid construction. Conversely, by hollow construction is meant the internal void that may exist between the inner and outer surfaces, depending on how it is manufactured. Such constructions may resemble a thin-walled hollow bulb that has been collapsed to expel most or all of the air, and indeed that is how initial prototypes were produced and tested. Compound constructions that have a viscous shape-conforming filling, like some shoe insoles and other common pressure pads, or that incorporate embedded materials to control physical characteristics like deformation or stretch, are also possible. An especially soft and pliable retainer that has an inelastic thread or fabric embedded in the material surrounding its hole to prevent the retainer from releasing too easily from an engaging device is one example of such a compound construction. All will be possible to manufacture economically in mass using ordinary molding techniques with flexible or resilient materials like common synthetic latex, silicone, polyurethane or nitrile rubbers. It should go without saying, like with any embodiment of this invention, that the only overriding criteria will be that the materials be sufficiently bio-compatible with the human body to prevent adverse bodily reactions during normal use. Such materials will not be difficult to find.

Production of the cup embodiments in a range of sizes will here too be intrinsic to their successful deployment and use. Unlike disk embodiments, where generally it will be desirable to use the largest retainer of size F that can comfortably pass thru the urethral orifice and hence where manufacturing them in closely spaced sizes to exacting tolerances will be desirable, sizing of cup embodiments will however likely not be as important. Users will generally find, due to the typically much larger outside perimeter P that can be achieved in a cup embodiment of any particular size F needed to allow it to pass thru the urethral orifice (the compressed French size F shown in FIG. 10), that there will be a broad choice of sizes that will all effectively perform the function of retaining whatever engaging device is employed and that the effectiveness of retention, which depends on the retainer being of a certain minimum size P, often will be of less concern than simple comfort while in use. As such it will usually not be necessary to select the largest possible F size, nor correspondingly to manufacture the cup embodiments in many closely spaced F sizes. Indeed, given that cup embodiments will have so much greater latitude in the F sizes that can be used while still maintaining retentive effectiveness, and because they can be manufactured so cheaply, it may not even be advantageous to adopt a standardized sizing convention like that suggested for disk embodiments. It may be preferable instead to simply size them in coarse increments like small, medium, large, etc. according to their P size and sell them in sets so that the user can simply choose the size that suits him best by trial and error. To facilitate use with existing common devices like catheters and stents, it is however likely that standardizing the diameter D of the hole (when one is provided), using a convention like the French system, will still be desirable.

Disk or cup embodiments of the invention rely on cooperation with a keeper or other device with the functional attributes of a keeper that must be separately inserted into the urethra and then engage the retainer for the retainer to be effectual or have any practical use. In certain applications this may leave some things to be desired, particularly when a simple effective seal and little more is needed to occlude the urethra, or where the objective is merely to externally hold the penis in a certain position. In a third basic embodiment of the invention, useful for occlusion and/or for fixating the penis to external devices without requiring the complication of a separate keeper or any other device that resides internally, the retainer is a hollow bulb-shaped structure that is retained within the fossa navicularis under the same principles that apply to its disk and cup counterparts, but that has integral to it at least one tubular appendage so that it remains accessible and operational from outside the penis at all times, thereby essentially comprising a retainer with the built-in functional attributes of a keeper all-in-one.

In its bulb embodiment, the bulbous structure is made of flexible but for all practical purposes inelastic material(s) which give it specific shape-modification and shape-retention characteristics so that it is capable of collapsing and folding onto itself when a suction pressure is applied to the interior thereof via the tubular appendage, and/or when it is laterally compressed from the outside, to thereby reduce its French perimeter in silhouette enough to facilitate easy passage through the external urethral orifice without requiring distension thereof or exerting significant pressure or friction thereon, and so that when the compressive force or suction pressure is released or a fluid like air or water is introduced via the tubular appendage, it rebounds or is restituted to its natural predetermined size and shape with the necessary material and physical attributes to resist deformation and dislocation from the fossa navicularis when any fluid pressures inside the urethra or external forces via the tubular appendage are subsequently applied thereto, with just enough pliability and/or shape conformance to form a comfortable sealing relationship with the urethral wall.

The bulb embodiment's distinction from ordinary elastic balloons and other inflatable devices like Foley catheters is that its volumetric capacity and shape in its normal un-collapsed or uncompressed state, i.e. in its operational state, are essentially predetermined and unvarying and, once in place, it relies either on the structural and resilient material characteristics of the retainer alone, or on the relative inelasticity of the retainer wall working against internal pressures, or on a combination of both, to maintain its size/shape and resist deformation, thereby through size and shape pre-selection alone, and not through adjustment of internal fluid pressure or volume, allowing precise fit and limiting the pressure (if any) it exerts on the urethral wall in the absence of other applied forces. It can collapse, but not expand beyond an essentially fixed size and shape. Except when made of very thin materials that depend on incompressible fluids or inflationary pressure for shape retention, the bulb embodiment will typically be designed to derive much of its ability to withstand strong external push or pull forces or pressures inside the urethra from its inherent bulb-like structural shape, like an egg shell can withstand forces that belie the fragility of its wall, giving the retainer the ability to be easily withdrawn from the penis with only a small fraction of the force that it can normally resist by simply first collapsing the retainer through suction or externally applied pressure to defeat the inherent structural strength of the bulb shape and thereby reduce pressures exerted on the urethral orifice during insertion or removal to those resulting from simple flexure of the resilient material as it passes through. Of course, if made of very thin non-elastic materials that constitute nothing more than a limp bag when vacated, pressure or friction on the urethral orifice during insertion or removal can be avoided entirely, but those types of bulb embodiments require a means of maintaining internal pressure and may not always be practical or desirable.

Referring to FIG. 11 which illustrates a sectional view through a central plane coincident with the axis of a representative basic example, in its typical bulb embodiment the retainer can be characterized as a bulbous hollow object combining, in virtually any combination, elements of spherical, ellipsoid, cylindrical and/or stylized rain-drop shapes that need not even be perfectly symmetrical (a short cylinder with spherical ends is shown), with a low-friction proximal or outer surface 10 that may either be smooth or ribbed or grooved and an inner surface 20 that also may either be smooth or ribbed or grooved (smooth surfaces are shown), with a thickness “T” between the inner and outer surfaces that, aside from any ribs or grooves, also may either be uniform (as shown) or may vary so as, in combination with any ribs or grooves that are present, to induce the bulb to collapse in a particular desired fashion when squeezed or when suction pressure is applied and/or to resist deformation when certain expected external forces are applied. External ribs or grooves may also be used to mitigate possible suction-cup like action and relieve external vacuum between the bulb and the urethral wall that may be created when the bulb is suddenly collapsed to a cup shape preparatory to removal, which some prototype tests have shown to be a potential source of discomfort when suction is used to collapse the bulb.

Integral to their fundamental form, bulb embodiments will typically also be characterized by a relatively slender flexible tube (rigid tubes are also possible) of outer diameter “D1” and wall thickness “T1”, centered on its axis with its lumen extending from the inner surface 20 of the bulb to the atmosphere, that tube being either monolithically formed as-one with the bulb (as shown) or of a different material and/or separate structure that is permanently bonded thereto. Technically there is no limit to how long this tubular appendage can be and its diameter D1 can also be almost as large as the targeted external urethral orifice itself, but designers will likely find it preferable to keep the tube's diameter D1 and its wall thickness T1 only as large as needed to permit collapsing the bulb and to prevent the tube from collapsing first when a suction pressure is applied thereto and also of just sufficient length and structural strength such that, when the retainer is positioned in the fossa navicularis, the tube remains readily accessible from outside the penis and is sturdy enough for engagement and use by cooperating external devices.

In operation, bulb embodiments may depend on ancillary devices to facilitate their use, such as a means for evacuating or filling the bulb, for maintaining a desired pressure within the bulb, or for externally fixating the retainer and hence the penis with respect to space or other objects. For ancillary purposes such as these, a fitting, for example like that of feature 140 in FIG. 11, may be incorporated on the distal end of the tube to facilitate use, but neither such fittings nor the ancillary devices that attach to them are intrinsic or essential to the invention and should not be confused with the keepers or devices with the functional attributes of keepers that are essential to its other embodiments. In point of fact, bulb embodiments comprising nothing more than a bulbous element with an attached tube can be functional and fully operational in themselves and, whereas it will generally be preferable to use suction to collapse the retainer first, they may be designed to allow insertion or removal merely by physically compressing the bulb from outside the penis with the thumb and fingers to first defeat its structural bulb shape before inserting or withdrawing it through the external urethral orifice. In this regard, the integrated tube is the only thing that is even remotely comparable to a keeper (although technically it is not a keeper) and that is intrinsic and essential to the invention, to keep the retainer physically accessible from outside the penis and its interior in fluid communication with the atmosphere or external suction or pressure devices, and the fitting 140 of FIG. 11 is shown only to aid in its operational understanding.

To illustrate the bulb embodiment's versatility and how its basic concept can be extended to include other ancillary functions as well, FIG. 12 shows another representative example in which a second smaller tube of diameter “D2” and wall thickness “T2” resides within the first tube of diameter “D1” and wall thickness “T1”, leaving only enough space within the larger tube to preserve fluid communication with the interior of the bulb via the larger tube, the smaller tube extending through the bulb along its axis to the outer surface 10 on the bulb's far side such that, by being bonded there and open to the bulb's exterior, it provides a separate isolated passage through the bulb for the drainage of bodily fluids or for the introduction of medicines or other fluids like x-ray contrast media. In this variation, collapse or restitution of the bulb during the insertion/removal process, when performed by applying negative or positive fluid pressures to the interior of the bulb, will be achieved by isolating or temporarily plugging the inner tube. FIG. 12 shows an example of a fitting 150 that could be used to facilitate such operation. Like that of feature 140 in FIG. 11, it is included only for illustrative purposes and is not intrinsic to the invention.

From the examples of FIGS. 11 & 12 it is easy to see that the concept of the basic bulb embodiment can be applied to numerous different applications and modified or extended in many ways. By this token, bulb embodiments that incorporate more than one passage through the bulb for the introduction or drainage of fluids, whether those passages are nested or arranged side by side, are considered obvious and within the scope of the invention Likewise, bulb embodiments that incorporate small sensors, heating elements or light producing elements within or on the exterior of the bulb, or that incorporate electrical contacts on the surface or electrical conductors that pass through the bulb, are also considered to be obvious and within the scope of the invention and merely a predictable extension of its design and possible uses. By the same token, bulb embodiments that are incorporated into or permanently fixated to devices that reside intraurethrally and/or external to the penis are also equally obvious. The external tube fittings and the devices like valves, syringes or pumps to facilitate bulb embodiments' intended modes of operation, whether for single-tube or for multiple-tube configurations and whether the operating mode involves collapsing the bulb via suction or restituting its shape via positive pressure or both, will not be difficult to design or manufacture and are in any event only ancillary and not pertinent to the novelty of the invention. Any functional device or method will do without going beyond the scope of the invention. For example, an ordinary syringe or, even more simply, just a removable tube extension to facilitate application of suction by mouth, will likely be the easiest and most expedient ways to collapse the bulb for insertion or removal in most cases.

Thin-walled bulb embodiments that rely heavily on the maintenance of internal fluid pressure, such as by being pressurized by a gas or filled with an incompressible fluid like water and then sealed, to hold their predetermined size and shape, will generally be found to be the easiest and least stressful to insert through the external urethral orifice due to their thinner construction and lesser material resilience, but in their operative in situ state may tend to be harder, more rigid and less conforming (hence less comfortable) than thicker-walled constructions that rely solely on material resilience to hold their size and shape. Thick-walled bulb retainers that depend on material resilience alone to function will usually be softer and more deformable, offering more latitude for self-adjustment and conformity to the confines of the fossa navicularis, but their critical size F in the collapsed state (depicted in FIG. 10) will tend to be larger and their greater resilience in that state will impose more stress on the urethral orifice during the insertion or removal process, potentially offsetting that advantage. To explain: generally bulb embodiments will naturally collapse or be induced to collapse to a cup-like shape, similar to that of cup embodiments, when the interior air or other fluid is removed, requiring the user to then further fold and manually compress the bulb, the procedure of insertion at that point being essentially identical to that for cup embodiments as depicted in FIG. 9. With limited exceptions, both cup and bulb embodiments will thus inherently put more pressure and friction on the external urethral orifice than disk embodiments, especially during the removal process when the fingers cannot easily be used to keep the retainer compressed except by externally squeezing the penis itself, the pressure and friction generally being more pronounced for bulb embodiments that rely on material resilience than those that rely on internal pressures to maintain shape. Design tradeoffs will therefore here too be inevitable, and the invention is by the same token not limited to either of the two extremes, but rather may rely on both material resilience and internal pressure in varying proportion for shape retention and operation, governed only by the basic principles of design and operation and the fundamental modes of implementation described herein.

The manufacture of bulb embodiments will be very easy and economical, using familiar processes like dip-molding or blow-molding with common materials like synthetic latexes, silicones, polyurethanes or nitriles for the bulbs, reinforced if necessary by inelastic filaments or fabrics embedded therein, and perhaps using stronger materials like nylons or vinyls for the tubes, and is expected to be as inexpensive as producing baby soothers or the cheap artificial grape clusters often used for home decoration. By their very nature, their collapsed/compressed French size F which is critical to insertion will also typically place little constraint on the un-collapsed size P that can be achieved and users will find that they can effectively use bulb embodiments in a broad range of P sizes with little variation in performance, the only significant difference being slight variations in comfort. Like for cup embodiments, sizing will therefore likely not be a major issue. They can be produced so cheaply and have so much latitude in fitting that it probably will be sufficient to produce them merely in a coarse range of sizes (like small, medium, large, etc.) and sell them in sets in the same way suggested for cup embodiments, letting the user decide by trial and error what suits him best, rather than attempt to standardize and identify their size by some common and more precise convention like the French system and sell them individually, although the invention does not preclude manufacturers from doing so.

The keeper, that is needed for disk or cup embodiments, derives its novelty from the retainer and its function and purpose are twofold: one, to engage the retainer following the retainer's placement within the fossa navicularis, thereby maintaining the retainer's orientation and where necessary also its assemblage (if it has more than one part) within the fossa navicularis and thus making the retainer retentively functional; and two, by such engagement also making the retainer in turn useful with any cooperating device, internal, external or both, that incorporates the functional elements and attributes of the keeper or that may further engage the keeper.

Because one of the intentions of the invention is to allow disk and cup embodiments to be used with known devices from the prior art, and because in such use it is conceivable and indeed anticipated that some of those devices will be able to perform like a keeper without any physical modification whatsoever, it is necessary to narrow the definition of the keeper so that it does not read on anything from the prior art. To simply say it is a retainer-enabling device is not enough. Accordingly, the keeper is herein defined as comprising intraurethral elements and operational features of a cooperating device that are not just functionally but also physically identifiable as being specifically retainer-enabling, although retainer-enabling does not necessarily have to be their exclusive purpose, i.e. that enable the device to cooperatively function with a disk or cup embodiment of the retainer for the purpose of intraurethral fixation and/or occlusion, regardless of whether the device is ostensibly intended to be used or is actually used in that cooperative manner, provided those elements and operational features of the cooperating device are physically distinguishable from the prior art insofar as their retainer-enabling characteristics are concerned and their ability to enable use of said retainer is not just the result of mere happenstance or fortunate coincidence. By including the key phrase “not just functionally but also physically identifiable” in this definition, the intention is to clearly differentiate between existing devices from the prior art that by coincidence and without any physical design modification are able to perform the functions of the keeper and make opportunistic use of the retainer and those devices from the prior art that require physical modification to do so, the former being excluded and the latter falling within the scope and definition of the keeper.

The keeper will, by its definition, obviously be able to take myriad physical forms. Narrowing it down, it can be described as further comprising:
first means for the at-will engagement of a disk or cup embodiment of the retainer by the cooperating device, while that retainer is positioned within the fossa navicularis, so that the cooperating device is fixated relative to that retainer while so engaged;
second means for fixating that retainer's assemblage (if it is composed of more than one component) and/or its orientation relative to a preferred position within the fossa navicularis, so that that retainer's assemblage and/or its orientation with respect to that preferred position does not change while that retainer is engaged by the cooperating device; and
third means for the at-will disengagement of that retainer from the cooperating device, while that retainer is positioned within the fossa navicularis, so that that retainer is free to be disassembled (if it is composed of more than one component) and/or repositioned and to be removed from the fossa navicularis in a process that is separate from insertion or removal of the cooperating device.

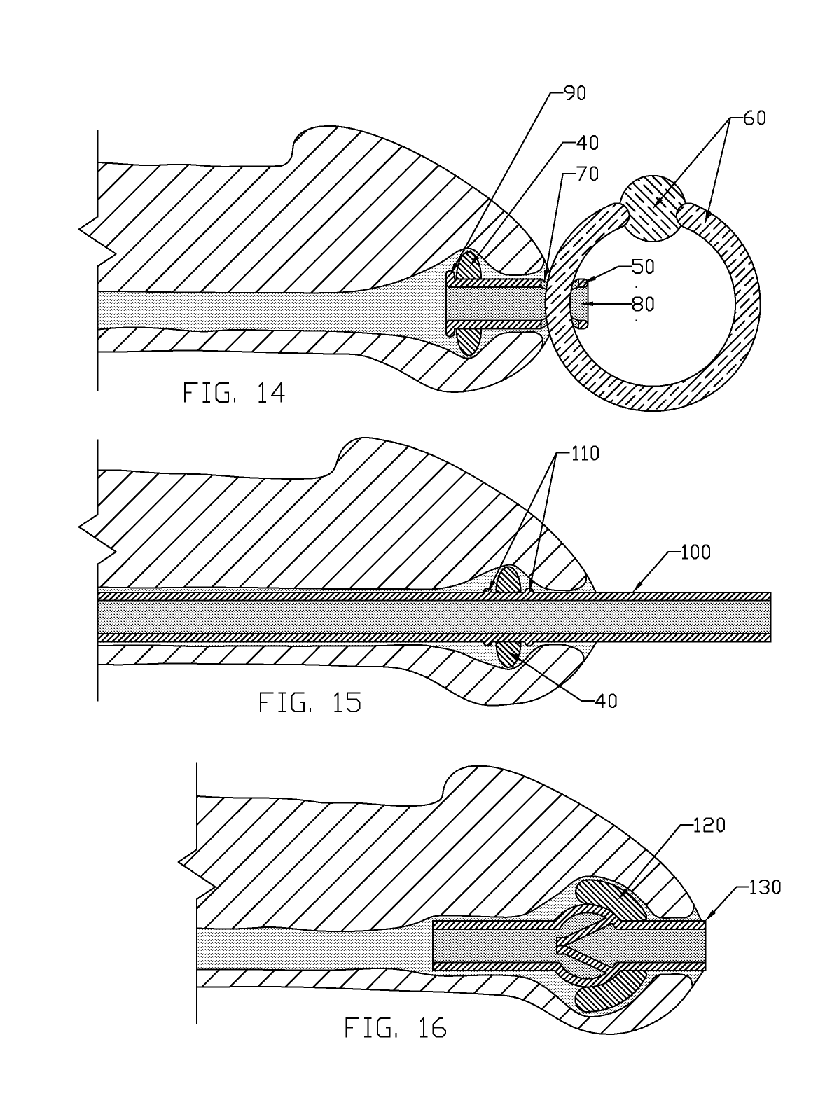

As its definition implies, the keeper will rarely, if ever, be a self-contained device, but more commonly part of a cooperating device in which the keeper's function and purpose is auxiliary and incidental to other objectives and functions of the device that incorporates it. The best way to physically visualize and fully understand what the keeper is and what it comprises, is by considering an example of a rudimentary cooperating device that incorporates and embodies it, such as the novel device shown in FIG. 14. In this example, the cooperating device is a simple stainless steel spout 50 that is shown being used in cooperation with a disk embodiment of the retainer 40 and that, in addition to its own features, has all the necessary characteristics of a keeper and hence is deemed to incorporate a keeper or be a keeper in addition to being a spout or whatever else it might be called. The spout's purpose and objective in this example is to mimic the physical appearance and emulate the function of a Prince Albert body piercing and it does so in this illustration by holding close to the penis a large decorative captive-bead ring 60, of the type often employed with such piercings, attachment of the ring being via the bilateral holes 70 located near the spout's outer end. Essentially, with the spout being barely visible and the ring held securely in place, it looks and functions very much like a Prince Albert piercing. The spout has an open lumen or axial bore 80 to permit micturition and ejaculation without removing the spout and retainer from the penis and quite possibly, provided the ring gauge is small enough, without even removing the ring from the spout. These features are however only incidental and not material to the spout's qualifying as incorporating a keeper or being a keeper.

What does make the spout shown in FIG. 14 qualify as incorporating or being a keeper, regardless of its own novelty and what it might be used for, is the fact that it depends on the retainer to stay inside the penis and to be of any use and that in this context is clearly and undeniably a retainer-enabling device unknown in the prior art. The spout is in this instance intended by its design to be inserted into the penis first, followed by the retainer, and then to be drawn back through the retainer with the aid of a string (dental floss being ideal for this purpose), or simply pushed back thru by externally manipulating the penis, the widened rim 90 at the distal end of the spout preventing the spout from pulling or pushing all the way thru the retainer and providing positive engagement of the retainer. This method of engagement corresponds to the first means that was noted above. Once in position, the barrel of the spout prevents the retainer from being distorted or rotating back to an orientation that would allow its expulsion or withdrawal from the penis, thus making the retainer itself functional and corresponding to the second means. The spout can then be used to attach the ring shown in FIG. 14, or other devices, to make itself useful. And by merely pushing the spout back out of the retainer with a pen or other suitable instrument, the two pieces can easily be individually squeezed back out of the penis in accordance with the third means that was noted above.

The spout shown in FIG. 14, for whatever purpose it might be used, is disclosed herein only as an illustrative example and falls within the scope and concept of the invention claimed herein only insofar as it qualifies as incorporating or being a keeper. Obviously, a spout like this can have many variations and serve numerous purposes, not just as a novel non-invasive alternative to body piercings for the attachment of jewelry and other sexual enhancements, but possibly also to occlude the urethra or to attach a collection device for the control of incontinence (as an alternative to catheters), to apply strong external traction or vibratory forces for therapeutic or sexually titillating purposes, or merely to hold the penis stationary for medical procedures like x-rays or adult circumcision. The example shows the spout being used with a disk embodiment, but a cup embodiment could also just as easily be used, the amount of tensile force that can be placed on the spout before it releases from the retainer, or before the entire assembly is dislodged from the penis, possibly being dependent on the material characteristics of the retainer (its elasticity) and the shape and dimensions of the widened end of the spout, allowing that force to be deliberately engineered and predetermined for safety if desired or needed, as was previously described. And just as obviously, the spout could also be enhanced or modified in many other ways, such as by incorporating a valve mechanism to regulate flows, or adding dedicated means to facilitate its attachment to other external devices besides or in lieu of a ring, none of those features being material to its qualification as a keeper. The spout of FIG. 14 being just one rudimentary example, it is expected there will be many devices that are able to take advantage of the retainer disclosed herein, and no doubt many of these cooperating devices will, to a degree, be novel in their own right, but that does not mean that they will not also qualify as incorporating or being a keeper under the scope and concept of the present invention, with all the protection that that entails.

Likewise, it is to be understood that the invention is not limited in scope by the method used to engage the retainer, whether or not the cooperating device is an existing appliance like a catheter or valve, or a novel new appliance like the spout of FIG. 14. Although a simple round hole in the retainer, sized to impede or prevent some portion of the keeper or other engaging device from freely passing through, coupled with a corresponding physical obstruction on the keeper or other device to impede or prevent such passage, such as the widened rim 90 of the spout shown in FIG. 14, is likely to be the most common means for engagement, other means are not excluded. These other means of engagement may include, but are not limited to, means that involve mechanically screwing the keeper into the retainer via engaging threads or that capture and hold the retainer through some sort of hook, key or other latch mechanism; prime candidates being means that engage or release the retainer in a manner similar to quick-release fluid couplings or other ball-lock mechanisms like the type commonly used to hold an ordinary mechanic's socket on a socket wrench or that are often found on detachable key chains. Many of these alternative means of engagement will be focused on being able to attach the cooperating device, i.e. the keeper, after the retainer is inserted, rather than by inserting the keeper into the urethra first, there being many potential applications where such action will be desirable. It is envisioned that one very strong and simple way of accomplishing this could be by employing today's very powerful rare-earth magnets and devising a keeper that incorporates an externally-operable plunger mechanism to prevent magnetic parts of the retainer and keeper from uncontrollably snapping together when they come within proximity of each other and to physically separate them without stress on the urethra when it comes time for removal. Such a retainer and keeper needn't be complex or difficult to make and could have many advantages. Without infringing on the potential novelty and patentability of such means of engagement, or for that matter infringing on that of any novel engaging device itself, that might in the future be invented, any device or method may be employed to engage the retainer in its various embodiments without going beyond the scope or concept of the present invention.

Notwithstanding that the invention's greatest value will likely come from its use with novel new devices, like the above spout, for purposes that were previously not thought to be feasible or practical, or that were not even imagined until now, it is also expected to be valuable in addressing old problems. FIG. 15 for example shows how the same disk embodiment 40 could be used to hold an ordinary rubbery catheter or stent 100 in place, particularly when something like an ordinary Foley catheter cannot be used. In this particular example the catheter or stent has been adapted to the retainer by the addition of two small annular ribs 110 molded into the surface to engage the retainer and hold it in place, those ribs clearly qualifying it as a keeper. With such simply modified catheters or stents, installation can be accomplished by inserting the appliance into the penis first, provided it is not too long, and then drawing it back through the retainer with the aid of a removable string or other suitable appendage after the retainer is inserted. This method would take advantage of the fact that an annular rib on a rubbery deformable tube will typically be much easier to pull, rather than to push, through the retainer, thus making engagement and disengagement relatively easy yet providing strong resistance to dislocation of the catheter or stent in normal use after it is in place. Alternatively, the catheter or stent might be further modified to accept a thin flexible plunger that stretches the tube and reduces the diameter of the annular ribs, allowing the catheter or stent to be inserted after the retainer is inserted. Or to avoid the necessity of annular ribs, the catheter or stent might be modified in its interior to incorporate a moveable tapered bushing that wedges the tube against the retainer from the inside at the desired position, also allowing the catheter or stent to be inserted after the retainer and allowing it to be removed without removing the retainer first, any of those physical modifications also qualifying it as a keeper.

These methods of engagement would all be considered to make the catheter or stent qualify as incorporating or being a keeper and fall within the scope of the invention. But merely using a retainer, or being able to use a retainer, will not automatically make the engaging device qualify as having or being a keeper. To qualify as having or being a keeper, and not just a device with the functional attributes of a keeper, the engaging device must incorporate physical elements and features whose reason for existence is at least in part to intentionally and purposefully enable use of the retainer and make the retainer functional. In the case of catheters and stents, and any other common existing device that might be used, those that are able to make opportunistic use of the retainer without physical modification, such as by merely selecting the retainer's hole to be a little smaller than the catheter or stent so that friction alone is enough to maintain engagement, may do so without being considered to incorporate or be a keeper or infringing on the invention.

An excellent hypothetical example where the engaging device would not be considered to incorporate or be a keeper, but where the retainer could be used to considerably improve its performance, is shown in FIG. 16. In this illustration a cup embodiment 120 is shown being used in cooperation with the urinary control valve 130 described in the Salama invention that was mentioned in the background, thereby overcoming the chief drawback of that invention: namely the fact that its globular valve must be small enough to pass through the external urethral orifice and that the retentive and sealing abilities of the device when used alone are thus very limited and questionable. In this example no modification of the Salama device would be needed, although the serrations described by Salama to increase friction and sealing ability would likely be redundant and unnecessary and have thus been eliminated in this illustration. Installation would be accomplished by inserting the valve first, like with the spout described above, and then drawing it back through the retainer with the aid of a removable string or other suitable appendage, or simply by external manipulation, after the retainer is inserted.

Notably, FIG. 16 is also a prime example of a situation that may be encountered often: where the retainer requires little or no resilience or structural integrity of its own, depending instead on the engaging globular valve for shape preservation and resistance to deformation and thus allowing it to be made of an especially soft compound, possibly even one with a viscous fluid interior, in this particular case assuring optimum comfort and much better sealing ability with no possibility of expulsion and without interfering with the intended mode of operation of the valve which is by external compression. Being larger than the valve and thus more easily manipulated, it can be surmised that the retainer would probably even assist valve operation and make it easier in this application. Soft pliable retainers like this, which cooperate with and depend on the keeper or other engaging device to hold their shape and resist any deformation that would lead to expulsion, will likely prove useful with many devices, both existing and novel.

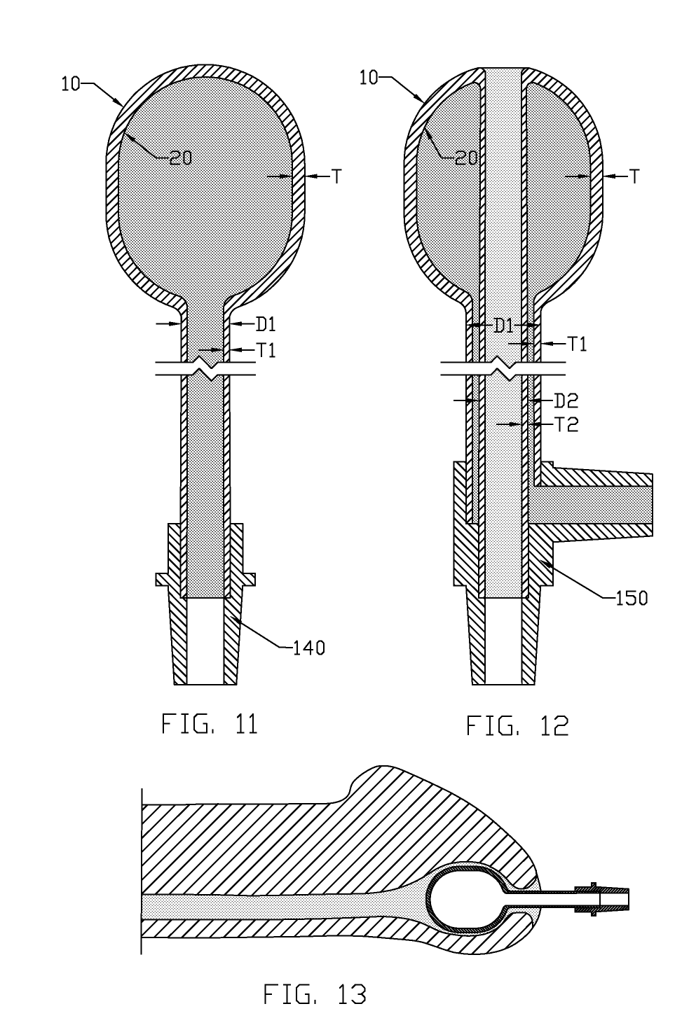

But if the control of urinary incontinence is the only objective, complex devices like the Salama invention are not even needed. A bulb embodiment of the invention, as shown in use in FIG. 13, can accomplish the same objective much more easily and effectively and has the major advantage that it does not require applying external pressure to the penis for operation. Prototype experiments have already shown that by simply collapsing it (for example by mouth suction with the aid of a temporarily attached tube) without removing the bulb from the penis, bulb embodiments can act as effective open/shut valves, with virtually no leakage, and permit the easy and convenient evacuation of urine whenever needed. Most notably, when collapsed, simple bulb embodiments have been observed to present very little obstruction to the flow of urine, thanks to the roominess of the fossa navicularis which then allows urine to bypass freely, facilitating quick and complete drainage, unlike more complex valves like the Salama invention that are susceptible to mechanical failure and whose lumen are smaller and more restrictive. Even without using suction, some prototype bulbs have been found to make micturition and drainage of the urethra not only possible, but notably also quite comfortable and convenient: simply by externally squeezing and manipulating the bulb with the fingers (probably with less pressure than the Salama would require) to distort the shape of the bulb and press it away from the external orifice.

In closure, it is to be understood that the particular physical forms, methods of engagement and the various applications of the embodiments of the invention described herein are given and shown by way of example only and are not to be construed as limitations of the invention. The scope and breadth of the invention may encompass numerous other embodiments and applications without departing from the gist of the invention, governed only by the fundamental design concepts and operating principles described herein and the claims that follow.

I claim:

1. An intraurethral retainer, for use with a cooperating device that incorporates a compatible keeper or that has the functional attributes of a compatible keeper, to facilitate fixation and/or occlusion of the human penis in joint use with that cooperating device, that action being its primary or foremost purpose, the intraurethral retainer comprising a separate device that is able to be placed within the fossa navicularis of a human penis separately from the cooperating device, and that, while it is operationally positioned within the fossa navicularis and while it is engaged by the cooperating device, is able to prevent or impede its own dislocation and correspondingly retain the cooperating device within the penis by utilizing the comparative (relative to its own size when operationally positioned within the fossa navicularis) constrictions of the urethral orifice and the corpus cavernosum urethrae as effective physical barriers to the intraurethral retainer's dislocation from its position.

2. The intraurethral retainer of claim 1, further comprising:
first means for sufficiently reducing the perceived French perimeter of the retainer, preparatory to the retainer's insertion into the fossa navicularis or its removal from the fossa navicularis and relative to the retainer's silhouette when viewed from a perspective that is in line with the urethra, such that while its perceived French perimeter from that perspective is so reduced, which is hereinafter referred to as the retainer's transitional state, passage of the retainer through the constriction of the external urethral orifice does not cause or necessitate dilation or distension of said orifice or exert significant pressure or friction on said orifice during such passage;
second means for sufficiently increasing the perceived French perimeter of the retainer, while it is positioned within the fossa navicularis and relative to the retainer's silhouette when viewed from a perspective that is in line with the urethra, such that while so positioned and while its perceived French perimeter from that perspective is so increased, which are hereinafter collectively referred to as the retainer's operational state, the comparative constrictions of the opposing external urethra orifice or the corpus cavernosum urethrae are small enough to function as effective physical barriers to the retainer's passage through them and obstruct the retainer's unintentional dislocation from its position within the fossa navicularis, and also such that, while the retainer is in its operational state, it is simultaneously ensured that the retainer's perceived French perimeter does not exceed that of the fossa navicularis and is still small enough for the retainer to comfortably reside within the fossa navicularis without, of its own accord and in the absence of any other force exerted on the retainer or the penis, exerting significant pressure or friction on the urethral wall; and
third means for the at-will engagement and disengagement of the retainer by the cooperating device, while the retainer is in its operational state, such that when the retainer is engaged a fixative relationship with the cooperating device exists that is sufficient both to maintain the retainer's operational state as well as to share in some measure with the cooperating device the retainer's retentive abilities to remain fixated within the fossa navicularis, and also such that when the retainer is disengaged, no fixative relationship with the cooperating device exists to prevent or obstruct the retainer's return back to its transitional state.

3. The intraurethral retainer of claim 1, further comprising a structure of one or more separate components that are compatibly sized and shaped such that:
one, when inserted into the urethra in a direction normal to the plane of its perceived least French perimeter when viewed in silhouette, the retainer or each of its components is small enough to individually be able to pass through the external urethral orifice and enter the fossa navicularis without causing distension or dilation of said orifice or exerting appreciable pressure or friction thereon and without necessitating deformation of the retainer or its individual components;
two, when the retainer is subsequently turned a number of degrees opposite to that plane inside the fossa navicularis, or when each of its components is subsequently turned a number of degrees opposite to that plane and its components are then assembled inside the fossa navicularis, its assemblage and/or orientation within the fossa navicularis hereinafter being termed as the retainer's operative state, the retainer's perceived French perimeter in silhouette, when viewed from a perspective that is in line with the urethra, is then large enough that the constrictions of the external urethral orifice or the corpus cavernosum urethrae function as effective physical barriers to the retainer's passage through them, but whose perceived French perimeter in that operative state and in that perspective is still small enough for the retainer to comfortably reside in the fossa navicularis without, of its own accord or by its mere presence, exerting significant pressure or friction on the urethral wall in the absence of any other force applied to the retainer or to the penis;
three, the retainer has means for the at-will engagement and disengagement by the cooperating device while it is within the fossa navicularis and while it is in the operative state, such that while it is so engaged, the retainer's operative state is maintained so as to facilitate the retainer and the cooperating device then being able to resist expulsion or dislocation from their position within the fossa navicularis by any force exerted on the cooperating device or the retainer itself that is not intended to cause or result in such expulsion or dislocation and thereby facilitate the retainer being cooperatively functional and operationally useful; and
four, when not engaged by the cooperating device, the retainer or each of its individual components can be turned back to the orientation it had when first inserted and individually removed from the fossa navicularis through the constriction of the external urethral orifice without, as during insertion, causing dilation or distension of said orifice or exerting appreciable pressure or friction thereon and without necessitating deformation of the retainer or its individual components.

4. The intraurethral retainer of claim 1, further comprising a structure that is compatibly sized and shaped and made of resilient and deformable materials such that:
one, when folded and/or compressed and then inserted into the urethra in a direction normal to the plane of its perceived least French perimeter when viewed in silhouette, the retainer is small enough and of low enough resilience to be able to pass through the external urethral orifice and enter the fossa navicularis without causing distension of said orifice or exerting appreciable pressure or friction thereon, allowing and withstanding that the retainer may, depending on its design and material composition, exert some reactive forces on the urethral orifice due to its resilience while it is attempting to unfold and/or decompress within the confines of said orifice as it is inserted and released;
two, after the retainer is permitted to rebound to its natural shape inside the fossa navicularis and when it is then turned a number of degrees opposite to the plane of its insertion, hereinafter termed as the retainer's operative state, the retainer's perceived French perimeter in silhouette, when viewed from a perspective that is in line with the urethra, is then large enough that the constrictions of the external urethral orifice or the corpus cavernosum urethrae function as effective physical barriers to the retainer's passage through them, but whose perceived French perimeter in that operative state and in that perspective is still small enough for the retainer to comfortably reside in the fossa navicularis without, of its own accord or by its mere presence, exerting significant pressure or friction on the urethral wall in the absence of any other force applied to the retainer or to the penis;
three, the retainer has means for the at-will engagement and disengagement by the cooperating device while it is within the fossa navicularis and while it is in the operative state, such that while it is so engaged, the retainer's operative state is maintained so as to facilitate the retainer and the cooperating device then being able to resist expulsion or dislocation from their position within the fossa navicularis by any force exerted on the cooperating device or the retainer itself that is not intended to cause or result in such expulsion or dislocation and thereby facilitate the retainer being cooperatively functional and operationally useful; and
four, when not engaged by the cooperating device, the retainer may be turned back to the orientation it had immediately following insertion and may be removed from the fossa navicularis through the constriction of the external urethral orifice without causing dilation or distension of said orifice or exerting appreciable pressure or friction thereon, allowing and withstanding that the retainer may, depending on its design and material composition, exert some reactive forces on the urethral orifice due to its resilience while the constriction of said orifice forces it back to a folded and/or compressed state as it is being squeezed out or withdrawn.

5. An intraurethral retainer, to facilitate fixation and/or occlusion of the human penis, comprising:
one, a hollow bulbous structure that is compatibly sized and shaped and that is sufficiently flexible and of low enough resilience such that it is capable of collapsing and being folded when a suction pressure is applied to the interior thereof and/or when it is compressed from the outside, its resilience and ability to collapse and to be folded being sufficient enough to reduce its perceived least French perimeter in silhouette to the point that it can pass through the external urethral orifice and enter the fossa navicularis without requiring distension or dilation of said orifice or exerting significant pressure or friction thereon, and whose material characteristics and construction are also such that when the compressive force or suction pressure is released or when a fluid like air or water is introduced under pressure to its interior, the hollow bulbous structure rebounds or is restituted to its predetermined size and shape, the hollow bulbous structure's perceived French perimeter in silhouette, when viewed from a perspective that is in line with the urethra, then being large enough that the constrictions of the external urethral orifice or the corpus cavernosum urethrae function as effective physical barriers to the hollow bulbous structure's passage through them, but whose perceived French perimeter in that perspective is still small enough for the hollow bulbous structure to comfortably reside in the fossa navicularis without itself exerting significant pressure or friction on the urethral wall in the absence of any other force applied to the retainer or penis, the material characteristics and construction of the hollow bulbous structure furthermore being such that its uncompressed shape and maximum volumetric size are not materially influenced by any internal working fluid pressure that might be applied and such that it has the structural and physical attributes to resist deformation and dislocation from the fossa navicularis when any fluid pressures inside the urethra or external forces are subsequently applied thereto, with enough pliability and/or conforming shape to form a sealing relationship with the urethral wall; and
two, an appendage of small enough French size to comfortably reside within the external urethral orifice of a human penis without causing distention or dilation thereof and comprising a hollow tubular structure that is either monolithically formed as-one with the hollow bulbous structure or that is of a different material and/or separate structure that is permanently connected thereto, with the lumen of the hollow tubular structure extending in open fluid communication from the inner surface and void of the hollow bulbous structure to the atmosphere, the material composition and/or dimensions of the hollow tubular structure being such that it does not impede the collapse, folding and insertion of the hollow bulbous structure into the fossa navicularis and also such that when suction pressure is applied to the interior of the hollow tubular structure, it remains in open fluid communication with the interior of the hollow bulbous structure and does not collapse before the hollow bulbous structure collapses, or when positive pressure is applied to the interior of the hollow tubular structure to restitute and/or maintain the hollow bulbous structure's intended operational state, the hollow tubular structure is able to withstand working pressures and does not burst or materially expand beyond its predetermined size, and furthermore the material composition and/or dimensions of the hollow tubular structure being such that, when the hollow bulbous structure is positioned in the fossa navicularis, a portion of the appendage comprising the attached hollow tubular structure remains readily accessible from outside the penis and is sturdy enough for engagement by any cooperating external devices that may be used to exert working pressures or other forces on the hollow bulbous structure.

6. The intraurethral retainer of claim 5, wherein the hollow bulbous structure and appendage comprise further means for the addition of at least one open lumen through said retainer for the drainage-from or introduction-to the urethra of fluids.

7. The intraurethral retainer of claim 5, wherein the hollow bulbous structure and the appendage comprise further means for the addition of at least one extra hollow tubular structure for the exchange of fluids within the hollow bulbous structure, such as may be desired for heating or cooling within the hollow bulbous structure.

8. The intraurethral retainer of claim 5, wherein the hollow bulbous structure and the appendage comprise further means for the addition of at least one electrical conductor that passes from outside the penis through said retainer to the interior of the urethra.

9. The intraurethral retainer of claim 5, wherein the hollow bulbous structure and the appendage comprise further means for the incorporation of electrical contacts and/or any of various pulse, temperature, pH, blood-pressure and other biological sensors, as well as for incorporation of heat-producing, light-producing or other radiant elements, within or on the exterior surface of the hollow bulbous structure.

10. An intraurethral keeper, for incorporation into a cooperating device to enable said device to be cooperatively used with the intraurethral retainer of claim 1, or with the intraurethral retainer of claim 2, or with the intraurethral retainer of claim 3 or with the intraurethral retainer of claim 4, the intraurethral keeper comprising integral elements and operational features of said device that are not just functionally but also physically identifiable as being specifically retainer-enabling, although retainer-enabling does not necessarily have to be their exclusive purpose, i.e. that enable said device to cooperatively function with any of the intraurethral retainers of claim 1, 2, 3 or 4 for the purpose of intraurethral fixation and/or occlusion, regardless of whether said device is ostensibly intended to be used or is actually used in that cooperative manner, provided those elements and operational features of the cooperating device are physically distinguishable from the prior art insofar as their retainer-enabling characteristics are concerned and their ability to enable use of said retainer is not just the result of mere happenstance or fortunate coincidence, the intraurethral keeper further comprising:
first means for the at-will engagement of said retainer by the cooperating device, while said retainer is positioned within the fossa navicularis, so that the cooperating device is fixated relative to said retainer while so engaged;
second means for fixating said retainer's assemblage (if it is composed of more than one component) and/or its orientation relative to a preferred position within the fossa navicularis, so that said retainer's assemblage and/or its orientation with respect to that preferred position does not change while said retainer is engaged by the cooperating device; and
third means for the at-will disengagement of said retainer from the cooperating device, while said retainer is positioned within the fossa navicularis, so that said retainer is free to be disassembled (if it is composed of more than one component) and/or repositioned and is free to be removed from the fossa navicularis in a process that is separate from insertion or removal of the cooperating device.

11. The intraurethral keeper of claim 10 wherein the intraurethral keeper comprises further means for helping to control said retainer's shape and to prevent undesirable deformation of said retainer while it is engaged by the keeper.
